(19) United States
(12) Patent Application Publication
(10) Pub. No.: US 2023/0348862 A1
(43) Pub. Date: Nov. 2, 2023

(54) IMMUNOENGINEERED PLURIPOTENT CELLS

(71) Applicant: The Regents of the University of California, Oakland, CA (US)

(72) Inventors: Sonja Schrepfer, Burlingame, CA (US); Tobias Deuse, Burlingame, CA (US)

(73) Assignee: The Regents of the University of California, Oakland, CA (US)

(21) Appl. No.: 18/127,936

(22) Filed: Mar. 29, 2023

Related U.S. Application Data
(63) Continuation of application No. 16/476,794, filed on Jul. 9, 2019.
(60) Provisional application No. 62/445,969, filed on Jan. 13, 2017.

(51) Int. Cl. C12N 15/113; C12N 15/52; C12N 15/85; C12N 5/074

(52) U.S. Cl. CPC C12N 5/0696; C12N 15/1138; C12N 15/52; C12N 15/85; C12N 2310/20; C12N 2501/599; C12N 2840/005; C12N 2840/007; C12Y 207/01021; C12Y 305/04001

(57) ABSTRACT

The invention provides pluripotent cells that are used therapeutically for regenerating tissues but avoid rejection by subjects that receive them. In particular, the invention provides hypo-immunogenic pluripotent cells that avoid host immune rejection. The cells lack major immune antigens that trigger immune responses and are engineered to avoid phagocytic endocytosis. The invention further provides universally acceptable “off-the-shelf” pluripotent cells and derivatives thereof for generating or regenerating specific tissues and organs.

### I. CROSS REFERENCE TO RELATED APPLICATIONS

This application is a continuation application of U.S. Application No. 16/476,794, filed Jul. 9, 2019, which is a U.S. National Phase Application of PCT/US2018/013688 filed Jan. 14, 2018 and claims the benefit of U.S. Provisional Application No. 62/445,969, filed on Jan. 13, 2017, which are incorporated herein by reference in their entirety.

### I.I. SEQUENCE LISTING

The instant application contains a Sequence Listing which has been submitted electronically in XML format and is hereby incorporated by reference in its entirety. Said XML copy, created on Jun. 5, 2023, is named RUC005US2_SL.xml and is 28,800 bytes in size.

### II. FIELD OF THE INVENTION

Regenerative cell therapy is an important potential treatment for regenerating injured organs and tissue. With the low availability of organs for transplantation and the accompanying lengthy wait, the possibility of regenerating tissue by transplanting readily available cell lines into patients is understandably appealing. Regenerative cell therapy has shown promising initial results for rehabilitating damaged tissues after transplantation in animal models (e.g. after myocardial infarction). The propensity for the transplant recipient’s immune system to reject allogeneic material, however, greatly reduces the potential efficacy of therapeutics and diminishes the possible positive effects surrounding such treatments.

### III. BACKGROUND OF THE INVENTION

Regenerative cell therapy is an important potential treatment for regenerating injured organs and tissue. With the low availability of organs for transplantation and the accompanying lengthy wait, the possibility of regenerating tissue by transplanting readily available cell lines into patients is understandably appealing. Regenerative cell therapy has shown promising initial results for rehabilitating damaged tissues after transplantation in animal models (e.g. after myocardial infarction). The propensity for the transplant recipient’s immune system to reject allogeneic material, however, greatly reduces the potential efficacy of therapeutics and diminishes the possible positive effects surrounding such treatments.

Autologous induced pluripotent stem cells (iPSCs) theoretically constitute an unlimited cell source for patient-specific cell-based organ repair strategies. Their generation, however, poses technical and manufacturing challenges and is a lengthy process that conceptually prevents any acute treatment modalities. Allogeneic iPSC-based therapies are easier from a manufacturing standpoint and allow the generation of well-screened, standardized, high-quality cell products. Because of their allogeneic origin, however, such cell products would undergo rejection. With the reduction or elimination of the cells’ antigenicity, universally-acceptable cell products could be produced. Because pluripotent stem cells can be differentiated into any cell type of the three germ layers, the potential application of stem cell therapy is wide-ranging. Differentiation can be performed ex vivo or in vivo by transplanting progenitor cells that continue to differentiate and mature in the organ environment of the implantation site. Ex vivo differentiation allows researchers or clinicians to closely monitor the procedure and ensures that the proper population of cells is generated prior to transplantation.

In most cases, however, undifferentiated pluripotent stem cells are avoided in clinical transplant therapies due to their propensity to form teratomas. Rather, such therapies tend to use differentiated cells (e.g. stem cell-derived cardiomyocytes transplanted into the myocardium of patients suffering from heart failure). Clinical applications of such pluripotent cells or tissues would benefit from a “safety feature” that controls the growth and survival of cells after their transplantation.

The art seeks stem cells capable of producing cells that are used to regenerate diseased or deficient cells. Pluripotent stem cells (PSCs) may be used because they rapidly propagate and differentiate into many possible cell types. The family of PSCs includes several members generated via different techniques and possessing distinct immunogenic features. Patient compatibility with engineered cells or tissues derived from PSCs determines the risk of immune rejection and the requirement for immunosuppression.

Embryonic stem cells (ESCs) isolated from the inner cell mass of blastocysts exhibit the histocompatibility antigens that are mismatches with recipients. This immunological barrier cannot be solved by human leukocyte antigen (HLA)-typed banks of ESCs because even HLA-matched PSC grafts undergo rejection because of mismatches in non-HLA molecules that function as minor antigens. To date, preclinical success of PSC-based approaches has only been achieved in immunosuppressed or immunodeficient models, or when the cells are encapsulated and protected from the host’s immune system. Systemic immunosuppression as used in allogeneic organ transplantation, however, is not justifiable for regenerative approaches. Immunosuppressive drugs have severe side effects and significantly increase the risk of infections and malignancies.

To circumvent the problem of rejection, different techniques for the generation of patient-specific pluripotent stem cells have been developed. These include the transfer of a somatic cell nucleus into an enucleated oocyte (somatic cell nucleus transfer (SCNT) stem cells), the fusion of a somatic cell with an ESC (hybrid cell), and the reprograming of somatic cells using certain transcription factors (induced PSCs or iPSCs). SCNT stem cells and iPSCs, however, may have immune incompatibilities with the nucleus or cell donor, respectively, despite chromosomal identity. SCNT stem cells carry mitochondrial DNA (mtDNA) passed along from the oocyte. mtDNA-coded proteins can act as relevant minor antigens and trigger rejection. DNA and mtDNA mutations and genetic instability associated with reprograming and culture-expansion of iPSCs can also create minor antigens relevant for immune rejection. This previously unknown immune hurdle decreases the likelihood of successful, large-scale engineering of compatible patient-specific tissues using SCNT stem cells or iPSCs.

### IV. SUMMARY OF THE INVENTION

Hypoimmune pluripotent (HIP) cells were generated that evade rejection by the host immune system. Syncytiotrophoblast cells of the placenta were harnessed that form the interface between maternal blood and fetal tissue. MHC I or HLA-I and MHC II or HLA-II expression was reduced. CD47 was increased. This pattern of impaired antigen presentation capacity and protection from innate immune clearance evaded the host immune rejection. This was shown for HIP cells and particular ectoderm, mesoderm, and endoderm-derived cells into which the HIP cells were differentiated.

Thus, the invention provides a method of generating a hypo-immunogenic pluripotent stem cell comprising: eliminating the activity of both alleles of a B2M gene in an induced pluripotent stem cell (iPSC); eliminating the activity of both alleles of a CIITA gene in the iPSC; and increasing the expression of CD47 in the iPSC.

In a preferred embodiment of the method, the iPSC is human, the B2M gene is human, the CIITA gene is human, and the increased CD47 expression results from introducing at least one copy of a human CD47 gene under the control of a promoter into the iPSC cell. In another preferred embodiment of the in method, the iPSC is murine, the B2m gene is murine, the Ciita gene is murine, and the increased Cd47 expression results from introducing at least one copy of a murine Cd47 gene under the control of a promoter into the iPSC cell. In a more preferred embodiment, the promoter is a constitutive promoter.

In some embodiments of the methods disclosed herein, the disruption in both alleles of the B2M gene results from a Clustered Regularly Interspaced Short Palindromic Repeats)/Cas9 (CRISPR) reaction that disrupts both of the B2M gene alleles. In other embodiments of the method, the disruption in both alleles of the CIITA gene results from a CRISPR reaction that disrupts both of the CIITA gene alleles.

The invention provides a human hypo-immunogenic pluripotent (hHIP) stem cell comprising: one or more alterations that inactivate both alleles of an endogeneous B2M gene; one or more alterations that inactivate both alleles of an endogenous CIITA gene; and one or more alterations causing an increased expression of a CD47 gene in the hHIP stem cell; wherein the hHIP stem cell elicits a first Natural Killer (NK) cell response that is lower than a second NK cell response elicited by an induced Pluripotent Stem Cell (iPSC) that comprises said B2M and CIITA alterations but does not comprise said increased CD47 gene expression, and wherein the first and second NK cell responses are measured by determining the IFN-γ levels from NK cells incubated with either of the hHIP or iPSC in vitro.

The invention provides a Human hypo-immunogenic pluripotent (hHIP) stem cell comprising: one or more alterations that inactivate both alleles of an endogeneous B2M gene; one or more alterations that inactivate both alleles of an endogenous CIITA gene; and an alteration causing an increased expression of a CD47 gene in the hHIP stem cell; wherein the hHIP stem cell elicits a first T cell response in a humanized mouse strain that is lower than a second T cell response in the humanized mouse strain elicited by an iPSC, and wherein the first and second T cell responses are measured by determining the IFN-γ levels from the humanized mice in an Elispot assay.

The invention provides a method, comprising transplanting the hHIP stem cells disclosed herein into a human subject. The invention further provides the use of the hHIP stem cells disclosed herein for the preparation of a medicament for treating conditions requiring cell transplantations.

The invention provides a hypoimmunogenic pluripotent cell, comprising an endogenous Major Histocompatibility Antigen Class I (HLA-I) function that is reduced when compared to a parent pluripotent cell; an endogenous Major Histocompatibility Antigen Class II (HLA-II) function that is reduced when compared to the parent pluripotent cell; and a reduced susceptibility to NK cell killing when compared to the parent pluripotent cell; wherein the hypoimmunogenic pluripotent cell is less susceptible to rejection when transplanted into a subject as a result of the reduced HLA-I function, the reduced HLA-II function, and reduced susceptibility to NK cell killing.

In some embodiments, the hypoimmunogenic pluripotent cell is reduced by a reduction in ß-2 microglobulin protein expression. In a preferred embodiment, a gene encoding the ß-2 microglobulin protein is knocked out. In a more preferred embodiment, the ß-2 microglobulin protein has at least a 90% sequence identity to SEQ ID NO:1. In a more preferred embodiment, the ß-2 microglobulin protein has the sequence of SEQ ID NO:1.

In some embodiments, the HLA-I function is reduced by a reduction in HLA-A protein expression. In a preferred embodiment, a gene encoding the HLA-A protein is knocked out. In some embodiments, the HLA-I function is reduced by a reduction in HLA-B protein expression. In a preferred embodiment, a gene encoding the HLA-B protein is knocked out. In some embodiments, the HLA-I function is reduced by a reduction in HLA-C protein expression. In a preferred embodiment, a gene encoding the HLA-C protein is knocked out.

In another embodiment, the hypoimmunogenic pluripotent cells do not comprise an HLA-I function.

The invention provides a hypoimmunogenic pluripotent cell wherein the HLA-II function is reduced by a reduction in CIITA protein expression. In a preferred embodiment, a gene encoding the CIITA protein is knocked out. In a more preferred embodiment, the CIITA protein has at least a 90% sequence identity to SEQ ID NO:2. In a more preferred embodiment, the CIITA protein has the sequence of SEQ ID NO:2.

In some embodiments, the HLA-II function is reduced by a reduction in HLA-DP protein expression. In a preferred embodiment, a gene encoding the HLA-DP protein is knocked out. In some embodiments, the HLA-II function is reduced by a reduction in HLA-DR protein expression. In a preferred embodiment, a gene encoding the HLA-DR protein is knocked out. In some embodiments, the HLA-II function is reduced by a reduction in HLA-DQ protein expression. In a preferred embodiment, a gene encoding the HLA-DQ protein is knocked out.

The invention provides hypoimmunogenic pluripotent cells that do not comprise an HLA-II function.

The invention provides hypoimmunogenic pluripotent cells with a reduced susceptibility to macrophage phagocytosis or NK cell killing. The reduced susceptibility is caused by the increased expression of a CD47 protein. In some embodiments, the increased CD47 expression results from a modification to an endogenous CD47 gene locus. In other embodiments, the increased CD47 expression results from a CD47 transgene. In a preferred embodiment, the CD47 protein has at least a 90% sequence identity to SEQ ID NO:3. In a more preferred embodiment, the CD47 protein has the sequence of SEQ ID NO:3.

The invention provides hypoimmunogenic pluripotent cells comprising a suicide gene that is activated by a trigger that causes the hypoimmunogenic pluripotent or differentiated progeny cell to die. In a preferred embodiment, the suicide gene is a herpes simplex virus thymidine kinase gene (HSV-tk) and the trigger is ganciclovir. In a more preferred embodiment, the HSV-tk gene encodes a protein having at least a 90% sequence identity to SEQ ID NO:4. In a more preferred embodiment, the HSV-tk gene encodes a protein having the sequence of SEQ ID NO:4.

In another preferred embodiment, the suicide gene is an Escherichia coli cytosine deaminase gene (EC-CD) and the trigger is 5-fluorocytosine (5-FC). In a more preferred embodiment, the EC-CD gene encodes a protein having at least a 90% sequence identity to SEQ ID NO:5. In a more preferred embodiment, the EC-CD gene encodes a protein having the sequence of SEQ ID NO:5.

In another preferred embodiment, the suicide gene encodes an inducible Caspase protein and the trigger is a chemical inducer of dimerization (CID). In a more preferred embodiment, the inducible gene encodes a Caspase protein comprising at least a 90% sequence identity to SEQ ID NO:6. In a more preferred embodiment, the gene encodes a Caspase protein comprising the sequence of SEQ ID NO:6. In a more preferred embodiment, the CID is AP1903.

The invention provides a method for producing a hypoimmunogenic pluripotent cell, comprising reducing an endogenous Major Histocompatibility Antigen Class I (HLA-I) function in a pluripotent cell; reducing an endogenous Major Histocompatibility Antigen Class II (HLA-II) function in a pluripotent cell; and increasing the expression of a protein that reduces the susceptibility of the pluripotent cell to macrophage phagocytosis or NK cell killing.

In one embodiment of the method, the HLA-I function is reduced by reducing the expression of a β-2 microglobulin protein. In a preferred embodiment, the β-2 microglobulin protein expression is reduced by knocking out a gene encoding the β-2 microglobulin protein. In a more preferred embodiment, the β-2 microglobulin protein has at least a 90% sequence identity to SEQ ID NO:1.In a more preferred embodiment, the β-2 microglobulin protein has the sequence of SEQ ID NO:1.

In another embodiment of the method, the HLA-I function is reduced by reducing the expression of HLA-A protein expression. In a preferred embodiment, the HLA-A protein expression is reduced by knocking out a gene encoding the HLA-A protein. In another embodiment of the method, the HLA-I function is reduced by reducing the expression of HLA-B protein expression. In a preferred embodiment, the HLA-B protein expression is reduced by knocking out a gene encoding the HLA-B protein. In another embodiment of the method, the HLA-I function is reduced by reducing the expression of HLA-C protein expression. In a preferred embodiment, the HLA-C protein expression is reduced by knocking out a gene encoding the HLA-C protein.

In another embodiment of the method, the hypoimmunogenic pluripotent cell does not comprise an HLA-I function.

In another embodiment of the method, the HLA-II function is reduced by reducing the expression of a CIITA protein. In a preferred embodiment, the CIITA protein expression is reduced by knocking out a gene encoding the CIITA protein. In a more preferred embodiment, the CIITA protein has at least a 90% sequence identity to SEQ ID NO:2. In a more preferred embodiment, the CIITA protein has the sequence of SEQ ID NO:2.

In another embodiment of the method, the HLA-II function is reduced by reducing the expression of a HLA-DP protein. In a preferred embodiment, the HLA-DP protein expression is reduced by knocking out a gene encoding the HLA-DP protein. In another embodiment of the method, the HLA-II function is reduced by reducing the expression of a HLA-DR protein. In a preferred embodiment, the HLA-DR protein expression is reduced by knocking out a gene encoding the HLA-DR protein. In some embodiments of the method, the HLA-II function is reduced by reducing the expression of a HLA-DQ protein. In a preferred embodiment, the HLA-DQ protein expression is reduced by knocking out a gene encoding the HLA-DQ protein.

In another embodiment of the method, the hypoimmunogenic pluripotent cell does not comprise an HLA-II function.

In another embodiment of the method, the increased expression of a protein that reduces the susceptibility of the pluripotent cell to macrophage phagocytosis results from a modification to an endogenous gene locus. In a preferred embodiment, the endogenous gene locus encodes a CD47 protein. In another embodiment, the increased protein expression results from the expression of a transgene. In a preferred embodiment, the transgene encodes a CD47 protein. In a more preferred embodiment, the CD47 protein has at least a 90% sequence identity to SEQ ID NO:3. In a more preferred embodiment, the CD47 protein has the sequence of SEQ ID NO:3.

Another embodiment of the method further comprises expressing a suicide gene that is activated by a trigger that causes the hypoimmunogenic pluripotent or differentiated progeny cell to die. In a preferred embodiment, the suicide gene is a herpes simplex virus thymidine kinase gene (HSV-tk) and the trigger is ganciclovir. In a more preferred embodiment, the HSV-tk gene encodes a protein having at least a 90% sequence identity to SEQ ID NO:4. In a more preferred embodiment, the HSV-tk gene encodes a protein having the sequence of SEQ ID NO:4.

In another embodiment of the method, the suicide gene is an Escherichia coli cytosine deaminase gene (EC-CD) and the trigger is 5-fluorocytosine (5-FC). In a preferred embodiment, the EC-CD gene encodes a protein having at least a 90% sequence identity to SEQ ID NO:5. In a more preferred embodiment, the EC-CD gene encodes a protein having the sequence of SEQ ID NO:5.

In another embodiment of the method, the suicide gene encodes an inducible Caspase protein and the trigger is a specific chemical inducer of dimerization (CID). In a preferred embodiment of the method, the gene encodes an inducible Caspase protein comprising at least a 90% sequence identity to SEQ ID NO:6. In a more preferred embodiment, the gene encodes an inducible Caspase protein comprising the sequence of SEQ ID NO:6. In a more preferred embodiment, the CID is AP1903.

### V. BRIEF DESCRIPTION OF THE DRAWINGS

FIG. 1A shows the rationale for the novel hypoimmune pluripotent cells described herein. Fetuses are protected from “rejection” during pregnancy by fetomaternal tolerance. The cells have downregulated MHC class I expression. They also have downregulated MHC class II expression. They also have upregulated CD47. FIG. 1B shows that fetomaternal tolerance is mediated by syncytiotrophoblast cells. FIG. 1C shows that syncytiotrophoblast cells show no MHC I and II and high CD47 levels.

FIG. 2 shows murine induced pluripotent stem cells (miPSC) generated from C57BL/6 fibroblasts. Pluripotency was demonstrated by the reverse transcriptase polymerase chain reaction (rtPCR). Multiple mRNAs associated with pluripotency were detected in miPSC cell extracts but not in uninduced cells (parental murine fibroblasts).

FIG. 3 confirms the pluripotency of the miPSC cells. The C57BL/6 miPSC cells formed teratomas in syngeneic mice as well as BALB/c nude and scid beige mice. No teratomas were formed in immunocompetent allogeneic BALB/c mice.

FIG. 4 shows that when ß-2-microglobulin expression is knocked out in the miPSC cells, MHC-I expression cannot be induced by IFN-γ stimulation (right panel). As a control, the parent miPSC cells were stimulated with IFN-γ (left panel) and increased their MHC-I expression.

FIG. 5 shows that the miPSC/ß-2-microglobulin knockout further comprising a Ciita expression knockout (double-knockout) did not show any baseline MHC-II expression and could not be induced by TNF-α to express MHC-II.

FIG. 6A shows increased Cd47 expression from a transgene added to the ß-2-microglobulin/Ciita double-knockout (iPSC^{hypo} cells). FIG. 6B shows that the C57BL/6 iPSC^{hypo} cells survive in the allogeneic BALB/c environment but the parental iPSC cells do not.

FIG. 7 shows one embodiment of the invention. It shows a schematic diagram of the iPSC engineering that resulted in the hypoimmune pluripotent cells of the invention. To generate hypoimmune stem cells, first CRISPR-Cas 9 engineering was used to knock out both of the B2m alleles. Second, CRISPR-Cas 9 engineering was used to knock out both of the Ciita gene alleles. Third, a lenti-virus was used to knock in a Cd47 gene.

FIG. 8A Schematically depicts the role of B2m in the MHC I complex. A B2m knock-out depletes MHC I in mice or HLA-I in humans. FIG. 8B schematically shows that Ciita is a transcription factor that causes MHC II expression in mice or HLA-II expression in humans. A Ciita knockout depletes MHC II or HLA-II expression.

FIGS. 9A, 9B and 9C show that B2m-/- iPSCs lack MHC-I expression, B2m-/-Ciita-/- iPSCs lack MHC-I and MHC-II and B2m-/-Ciita-/-Cd47 tg iPSCs lack MHC-I and MHC-II and overexpress Cd47.

FIGS. 10A, 10B, 10C, 10D and 10E show mouse models of transplanted “wild type iPSCs” v. hypoimmune PSCs into allogeneic or syngeneic host mice. Here, the iPSCs were formed from C57BL/6 mice, and the allogeneic mice are BALB/c. FIG. 10A, “wild type iPSCs” only formed teratomas in syngeneic C57BL/6 mouse thighs. In contrast, an immune response was mounted in the allogeneic host mice (BALB/c) and no teratomas grew. FIG. 10B, “wild type iPSCs” formed teratomas in syngeneic C57BL/6 mice. FIG. 10C, the immune response prevented teratoma formation in allogeneic BALB/c. FIG. 10D compares the T cell response (IFN-γ and IL-4) to the iPSC in syngeneic and allogeneic hosts using a spot frequency assay (frequency of cells releasing IFN-γ and IL-4). IFN-γ and IL-4 release was very low in C57BL/6 hosts but dramatically increased in BALB/c hosts. FIG. 10E depicts the B cell responses in syngeneic and allogeneic hosts. The iPSCs were incubated with the serum of the host animals that had previously received iPSCs. Bound immunoglobulins were measured using flow cytometry. The mean fluorescence intensity (MFI) was significantly higher from serum taken from the allogeneic BALB/c recipient hosts.

FIGS. 11A, 11B, 11C, 11D and 11E show the partial effect of knocking out the B2m gene in the iPSCs described above. FIG. 11A, B2m-/- iPSCs grew in syngeneic C57BL/6 mouse thighs, forming teratomas due to the lack of an immune response, while a partial immune response was mounted in the allogeneic host mice (BALB/c); e.g. some of the transplanted cells survive. FIG. 11B, B2m-/- iPSCs formed teratomas in the syngeneic mice. FIG. 11C, a partial survival (60%) was achieved in the allogeneic hosts. FIG. 11D, differences in the T cell response (IFN-γ and IL-4) between the two hosts showed that a mild but detectable response of T cells against the B2m-/- iPSCs. FIG. 11E shows the B cell responses in the different host mice, showing the weaker immune response as compared to wild type iPSCs. There was still a significantly stronger immunoglobulin response after allogeneic transplantation of B2m-/- iPSCs into BALB/c when compared to syngeneic transplantation into C57BL/6. Thus, there was limited survival of the B2m-/- iPSCs in in allogeneic recipients.

FIGS. 12A, 12B, 12C, 12D and 12E show the increased partial effect of knocking out the B2m gene and the Ciita gene in iPSCs on cell survival in syngeneic and allogeneic host mice. FIG. 12A, B2m-/- Ciita-/- iPSCs formed teratomas in syngeneic C57BL/6 mouse thights due to the lack of an immune response, while a partial (but reduced as compared tothe B2m-/- iPSCs) immune response was mounted in the allogeneic host mice (BALB/c). FIG. 12B, B2m-/- Ciita-/- iPSCs formed teratomas in the syngeneic mice. FIG. 12C shows that some cellular grafts (91.7%) survive in allogeneic hosts. FIG. 12D, T cell response (IFN-γ and IL-4) differences between the two hosts showed a mildly higher IFN-γ response in allogeneic versus syngeneic recipients. FIG. 12E depicts the B cell responses in the different host mice. The weaker immune response was compared to wt iPSCs and B2m-/- iPSCs. A significant difference between allogeneic and syngeneic recipients was not observed. Overall, there was limited survival of the B2m-/- Ciita-/- iPSCs in allogeneic recipients that can be attributed to a measurable immune response).

FIGS. 13A, 13B, 13C, 13D and 13E show the complete effect of knocking out the B2m gene and the Ciita gene and knocking in the Cd47 transgene in iPSCs on cell survival in syngeneic and allogeneic host mice. FIG. 13A, B2m-/- Ciita-/- Cd47tg iPSCs teratomas grew both in syngeneic C57BL/6 and allogeneic host thighs. All of the transplanted cell grafts survived. FIG. 13B, B2m-/- Ciita-/- Cd47tg iPSCs formed teratomas in C57BL/6. FIG. 13C, 100% of cellular grafts survived in the allogeneic hosts. FIG. 13D shows the lack of T cell response (IFN-γ and IL-4) in allogeneic recipients. No difference between the two hosts was observed. FIG. 13E Depicts the lack of B cell responses in allogeneic recipients. No difference between the two hosts was observed. Thus, there was complete survival of the B2m-/- Ciita-/- Cd47tg iPSCs in allogeneic recipients. They were not immunogenic as they elicited no T cell or B cell response.

FIGS. 14A, 14B and 14C show that the B2m-/- Ciita-/- Cd47tg iPSCs (referred to as non-immunogenic pluripotent cells (HIP) cells) evaded the host immune system. FIG. 14A, stimulatory NK cell ligand expression did not increase in the HIP cells. A fusion protein that recognizes various ligands of the NK cell transmembrane protein NKG2D was used to assess the level of activatory ligands, which may activate cytolytic NK cell activity. Fusion protein binding to iPSCs thus is an overall parameter for their expression of activating NKG2D ligands. FIG. 14B, HIP cells did not make NK cells increase their CD107a expression, a marker for functional NK cell activity. In contrast, B2m-/- Ciita-/- iPSCs induced CD107a expression on NK cells and thus triggered their cytolytic function. FIG. 14C, IFN-γ Elispot assays with purified syngeneic NK cells from C57BL/6 mouse spleen showed no NK cell response elicited by HIP cells. Thus, NK cells were not activated to release IFN-γ. The spot frequency for HIP cells was not different from that of unstimulated NK cells (neg. control). Only B2m-/- Ciita-/- iPSCs resulted in significantly increased IFN-γ spot frequencies.

FIGS. 15A and 15B show additional data showing that the HIP cells evaded rejection or killing by the innate immune system due to the Cd47 transgene. An in vivo NK cell assay had a mixture of 50% iPSCs and 50% HIPs that were injected into the NK-rich peritoneum of syngeneic C57BL/6 (syngeneic) mice. Here, cytotoxicity is caused by NK cells. After 24 and 48 hours, peritoneal cells were recovered and sorted. FIG. 15A compares the iPSCs with B2m-/- Ciita-/- iPSCs (no Cd47 transgene). The B2m-/- Ciita-/- iPSCs were selectively killed by NK cells. FIG. 15B compares iPSCs with B2m-/- Ciita-/- Cd47 tg iPSCs (HIP cells). The HIP cells were not selectively killed by NK cells. The 50% ratio of HIP cells among peritoneal iPSCs was maintained, indicating no NK cell stimulation. Thus, while MHC-I and MHC-II knockouts rendered the cells highly susceptible to NK cell killing, the Cd47 overexpression removed stimulatory NK cell interaction.

FIG. 16 shows that the murine HIP cells of the invention displayed a normal murine karyotype.

FIGS. 17A, 17B and 17C show that the murine HIP cells of the invention retained pluripotency during the engineering process. Rt-PCR analysis of markers generally accepted to indicate pluripotency are shown (Nanog, Oct 4, Sox2, Esrrb, Tbx3, Tcl1, and actin as a loading control). The pluripotent markers were expressed throughout the three-step engineering process. FIG. 17A compares iPSCs, B2m-/- iPSCs, and murine fibroblasts (negative control). B2m-/- iPSC cells retained the pluripotency genes. FIG. 17B shows the same analysis but the B2m-/- Ciita -/- iPSCs. They retained the same pluripotency genes. FIG. 17C shows the same analysis but with the B2m-/- Ciita -/- Cd47 tg iPSCs (HIP cells). These cells retained the same pluripotency genes. In addition, histology images of teratomas that developed after transplantation of HIP cells into SCID beige mice show that cell types associated with ectoderm, mesoderm, and endoderm were identified. Immunofluorescence markers for all three germ layers were detected (data not shown). Cell morphology was correct for neuro-ectoderm, mesoderm and endoderm. Immunofluorescence staining for DAPI, GFAP, cytokeratin 8 and brachyury confirmed the pluripotency of the HIP cells.

FIGS. 18A, 18B and 18C show HIP cells differentiated into mesodermal lineage cells and lost their pluripotency markers. FIG. 18A shows the pluripotent markers in the HIP cells (labeled “mHIP”) were lost in the differentiated murine endothelial cells (labeled miEC”). FIG. 18B shows the pluripotent markers were retained in the HIP cells but not in the differentiated murine smooth muscle cells (labeled “miSMC”). FIG. 18C shows the pluripotent markers were retained in the HIP cells but not in the differentiated murine cardiomyocytes cells (labeled “miCM”). These results were confirmed by immunohistochemistry (data not shown). Endothelial cells were detecting using anti-CD31 and anti-VE-cadherin, smooth muscle cells were detected using anti-SMA and anti-SM22 antibodies, and cardiomyocytes were detected using anti-Troponin I and anti-sarcomeric alpha actinin antibodies).

FIGS. 19A and 19B show that the HIP cells were differentiated into the endoderm lineage Islet cells (iICs) that produced C-peptide and insulin. FIG. 19A, differentiation markers were not detected in HIP cells but were in the induced islet cells. FIG. 19B, the induced islet cells produced insulin. Immunohistochemistry staining for C-peptide confirmed these results (data not shown).

FIGS. 20A and 20B show the HIP cells differentiated into the ectoderm lineage. FIG. 20A shows the HIP cells in vitro and FIG. 20B shows the differentiated neuronal cells. Immunohistochemical staining with the neuroectodermal stem cell marker Nestin and Tuj-1 confirmed these results (data not shown).

FIGS. 21A, 21B and 21C show that the cells differentiated from the HIP cells retained the depleted MHC I and II phenotype and Cd47 overexpression. FIG. 21A compares MHC-I, MHC-II, and Cd47 expression between the mouse induced endothelial cells (“miEC”) and the B2m-/- Ciita -/- Cd47 tg miEC cells. FIG. 21B compares MHC-I, MHC-II, and Cd47 expression between the mouse induced smooth muscle cells (“miSMC”) and the B2m-/- Ciita -/- Cd47 tg miSMC cells. FIG. 21C compares MHC-I, MHC-II, and Cd47 expression between the mouse induced cardiomyocytes (“miCM”) and the B2m-/- Ciita /- Cd47 tg miCM cells.

FIGS. 22A, 22B and 22C shows that the endothelial cells differentiated from the HIP cells are non-immunogenic. FIG. 22A, transplantation of the C56BL/6 miECs syngeneic and allogeneic mice. miECs in allogeneic BALB/c recipient mice generated a pronounced immune response but not in syngeneic mice. This was evidenced by strong IFN-γ Elispot and immunoglobulin responses (FACS analysis) in BALB/c recipients (FIG. 22B). FIG. 22C, neither HIP nor miEC cells generated an immune response in syngeneic or allogeneic recipients.

FIGS. 23A, 23B and 23C shows that the mouse induced smooth muscle cells differentiated from the HIP cells are non-immunogenic. FIG. 23A, transplantation of the C56BL/6 miSMCs syngeneic and allogeneic mice. miSMCs in allogeneic BALB/c recipient mice generated a pronounced immune response but not in syngeneic mice. This was evidenced by strong IFN-γ Elispot and immunoglobulin responses (FACS analysis) in BALB/c recipients. FIG. 23C, neither HIP nor miSMC cells generated an immune response in syngeneic or allogeneic recipients.

FIGS. 24A, B and C shows that the mouse induced cardiomyocyte cells differentiated from the HIP cells are non-immunogenic. FIG. 24A, transplantation of the C56BL/6 miCMCs syngeneic and allogeneic mice. Figure miCMCs in allogeneic BALB/c recipient mice generated a pronounced immune response but not in syngeneic mice. This was evidenced by strong IFN-γ Elispot and immunoglobulin responses (FACS analysis) in BALB/c recipients (FIG. 24B). FIG. 24C, neither HIP nor miCMC cells generated an immune response in syngeneic or allogeneic recipients.

FIG. 25 shows that the differentiated cells (miECS, miSMCs, miCMs) derived from HIP cells evaded rejection via the innate immune system. An NK fusion protein assay showed that none of the three differentiated cells had increased expression of stimulatory NK cell ligands when compared to differentiated cells derived from miPSCs.

FIGS. 26A and 26B show that miECs derived from HIP cells of the invention evaded immune reaction and achieved long-term survival in an allogenic host. FIG. 26A, miEC grafts derived from miPSCs showed long-term survival in syngeneic recipients (C57BL/6) but were rejected in allogeneic recipients (BALB/c). FIG. 26B, miECs derived from HIP achieve long-term survival after transplantation in both syngeneic and allogeneic recipients.

FIG. 27: miECS derived from HIP cells organized to form vascular structures in allogenic hosts. After transplantation within a Matrigel matrix, over six weeks, the miECs organize in a three-dimensional manner to form vascular structures. These results were confirmed by immunofluorescence staining for luciferase and VE-cadherin; the miECs were transduced to express luciferase before transplantation. Survival was monitored via bioluminescence imaging and the transplanted cells were identified with immunofluorescence staining against luciferase (data not shown).

FIG. 28 shows that the human HIP cells displayed a normal human karyotype.

FIG. 29 show that human HIP cells maintained pluripotency during the engineering process. The hiPSCs (e.g. the starting cells, prior to the alterations of the invention) and the HIP cells of the invention both have expression of the pluripotency genes (NANOG, OCT4, SOX2, DPPA4, hTERT, ZFP42, and DEMT3B; G3PDH served as a loading control) using PCR assays. Immunofluorescent staining confirmed this finding as the cells express TRA-1-60, TRA-1-81, Sox2, Oct4, SSEA-4 markers, and alkaline phosphatase (data not shown).

FIGS. 30A and 30B show that transplanted human HIP cells into humanized allogeneic mice did not cause an immune response. FIG. 30A shows that T cells did not respond to the transplanted HIP cells as measured by IFN-γ production or IL-5 in Elispot assays. In contrast, transplanted iPSCs did. FIG. 30B shows that only iPSCs caused a strong antibody response in flow cytometry. The HIP cells did not.

FIGS. 31A, 31B, 31C, and 31D show that the human HIP cells were differentiated into the mesodermal lineage. FIG. 31A shows the morphology of a human HIP cell. FIG. 31B shows the HIP-derived endothelial cells stained with CD31, VE-cadherin, and DAPI as a control. FIG. 31C shows the HIP-derived cardiomyocytes stained with a-sarcomeric actinin, Troponin 1, and DAPI as a control. FIG. 31D shows premature vessel formation by the HIP-derived endothelial cells. HIP-derived cardiomyocytes were observed beating (data not shown).

FIGS. 32A and 32B show that transplanted human endothelial cells derived from human HIP cells did not cause an immune response in allogeneic humanized mice. FIG. 32A, hiECs mounted a significant T cell response in IFN-γ and IL-5 Elispot assays whereas hiECs derived from human HIP cells did not. FIG. 32B, shows the B cell response in flow cytometry. Only the hiECs generated a significant immunoglobulin binding as measured by mean fluorescence intensity (MFI).

FIGS. 33A and 33B show that the transplantation of human cardomyocytes derived from human HIP cells did not result in an immune response in allogeneic humanized mice. FIG. 33A shows the differences in T cell responses for “wild type” hiCMs versus the B2M-/- CIITA-/- CD47tg HIP cells in IFN-γ and IL-5 Elispots. FIG. 33B shows the B cell response in flow cytometry. Only the “wild type hiCMs” generated a significant immunoglobulin loading of hiECs, as measured by mean fluorescence intensity (MFI).

FIGS. 34A, 34B, 34C and 34D show that the human HIP cells of the invention evaded rejection of the innate immune system. NK cells were isolated from BALB/c mice using Magnetically Activated Cell Sorting (MACS). 5×10⁶ stimulator cells (C57BL/6 iPSC derivatives, either iEC, iSMC, or iCM and either B2M-/- CIITA-/- or B2M-/- CIITA-/- CD47 tg), were incubated with 5×10⁶ MACS-sorted NK cells in an IFN-γ Elispot plate. After 24 hours, the spot frequency was determined with an Elispot reader. All three B2M-/- CIITA-/-derivatives induced a strong NK response. All three B2M-/- CIITA-/- CD47 tg derivatives, however, did not induce any NK cell response and their spot frequency was not statistically different from negative controls (isolated NK cells not incubated with a stimulatory cell). FIG. 34A shows endothelial cells. FIG. 34B shows smooth muscle cells. FIG. 34C shows cardiomyocytes. FIG. 34D shows a YAC-1 mouse lymphoma positive control.

FIGS. 35A, 35B and 35C show the innate immune response (or lack thereof). A mixture of 50% wt derivative (5×10⁶ cells) and 50% of either C57BL/6 B2m-/- Ciita-/- or B2m-/- Ciita-/- Cd47 tg derivative (5×10⁶ cells) was prepared. The cells were stained with 10 µM CFSE staining for 10 min and resuspended in 500 µl saline. The cell mixture was then injected into the NK-rich peritoneum of C57BL/6 (syngeneic) mice. In this syngeneic model, all cytotoxicity is caused by NK cells. After 48 h, peritoneal cells are recovered and sorted and their ratio was calculated. wt and engineered cells were identified by MHCI staining in FACS. FIG. 35A shows endothelial cells. FIG. 35B shows smooth muscle cells. FIG. 35C shows cardiomyocytes.

FIGS. 36A, 36B and 36C shows genetic engineering of human iPSCs verified by FACS. The lack of HLA I and HLA II was confirmed in B2M-/- CIITA-/- hiPSCs. Additionally, B2M-/- CIITA-/- CD47 tg showed a high CD47 expression. FIG. 36A shows the HLA I results. FIG. 36B shows the HLA II results. FIG. 36C shows the CD47 results.

FIGS. 37A and B show that the immune phenotype was maintained after differentiation of B2M-/- CIITA-/- CD47 tg iPSCs. When compared to unmodified wt derivatives, FACS analysis showed that B2M-/- CIITA-/- CD47 tg derivatives lacked HLA I and HLA II and overexpression of CD47. FIG. 37A shows endothelial cells and FIG. 37B shows cardiomyocytes.

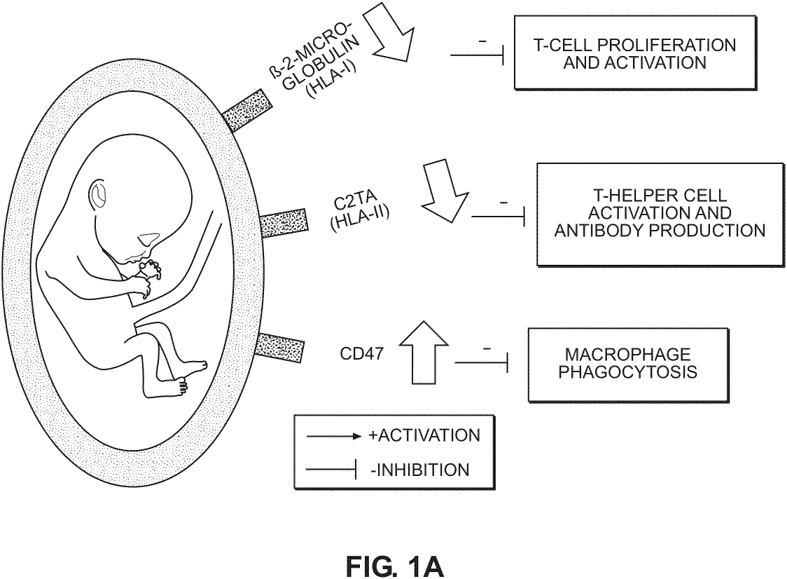

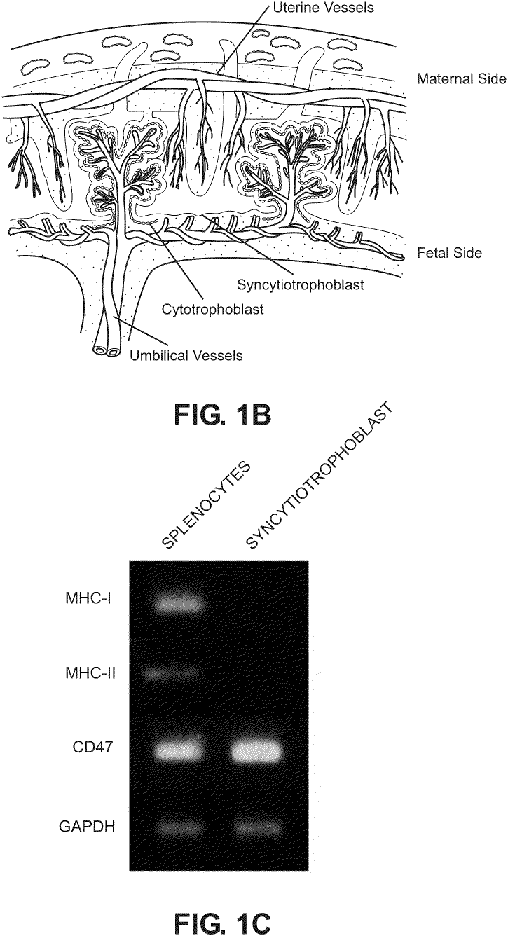

### VI. DETAILED DESCRIPTION OF THE INVENTION

### A. Introduction

The invention provides HypoImmunogenic Pluripotent (“HIP”) cells that avoid host immune responses due to several genetic manipulations as outlined herein. The cells lack major immune antigens that trigger immune responses and are engineered to avoid phagocytosis. This allows the derivation of “off-the-shelf” cell products for generating specific tissues and organs. The benefit of being able to use human allogeneic HIP cell derivatives in human patients results in significant benefits, including the ability to avoid long-term adjunct immunosuppressive therapy and drug use generally seen in allogeneic transplantations. It also provides significant cost savings as cell therapies can be used without requiring individual treatments for each patient. Recently, it was shown that cell products generated from autologous cell sources may become subject to immune rejection with few or even one single antigeneic mutation. Thus, autologous cell products are not inherently non-immunogenic. Also, cell engineering and quality control is very labor and cost intensive and autologous cells are not available for acute treatment options. Only allogeneic cell products will be able to be used for a bigger patient population if the immune hurdle can be overcome. HIP cells will serve as a universal cell source for the generation of universally-acceptable derivatives.

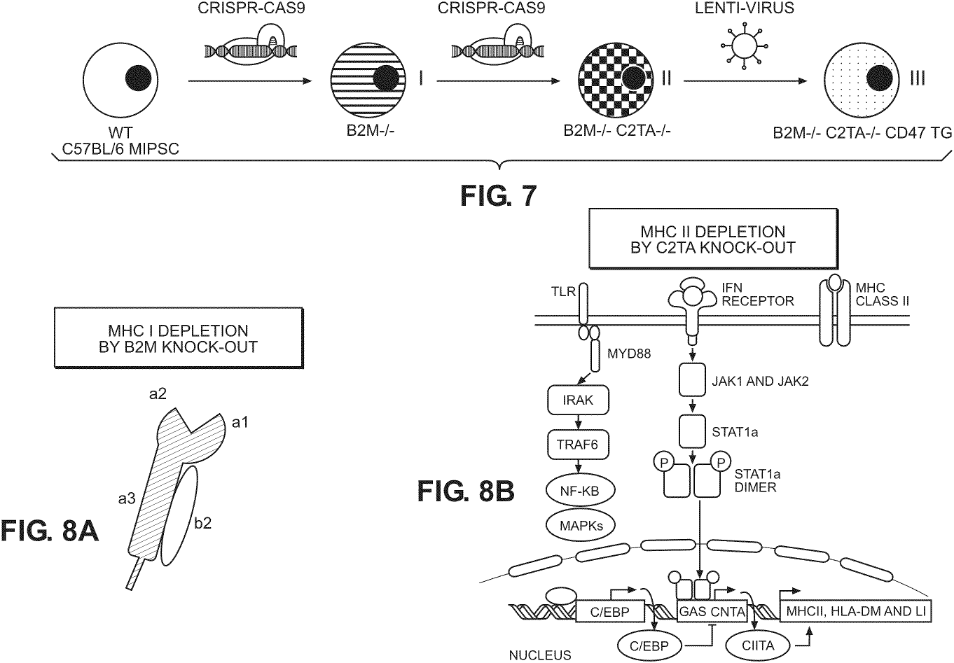

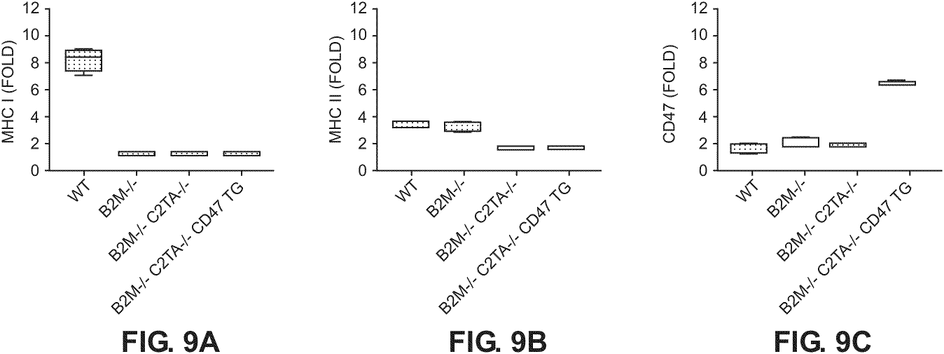

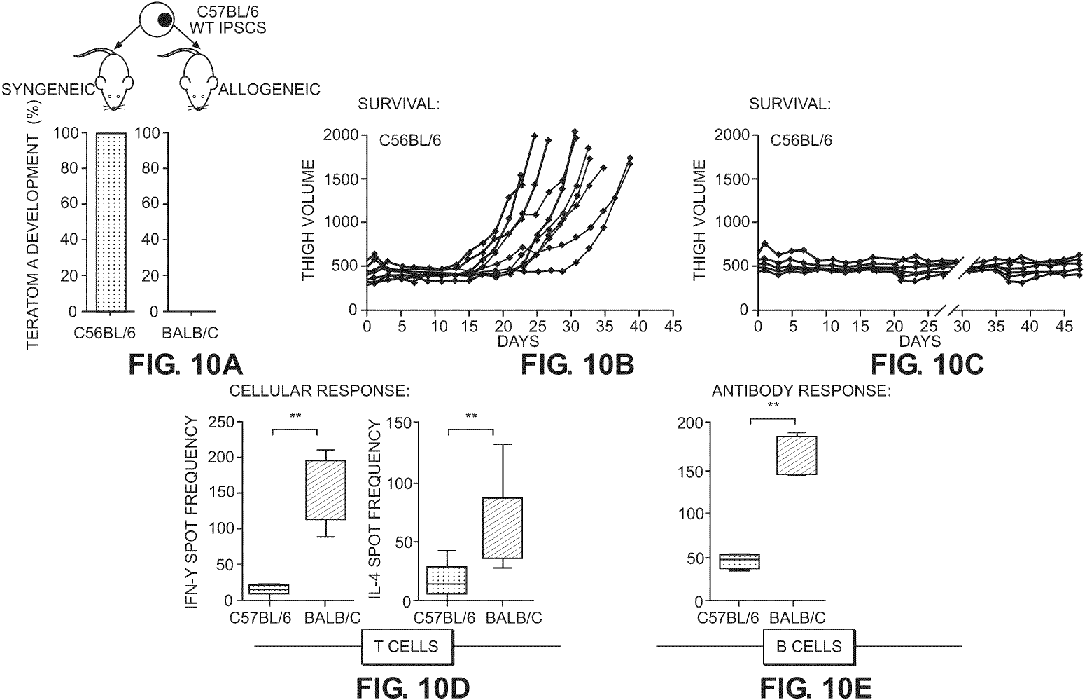

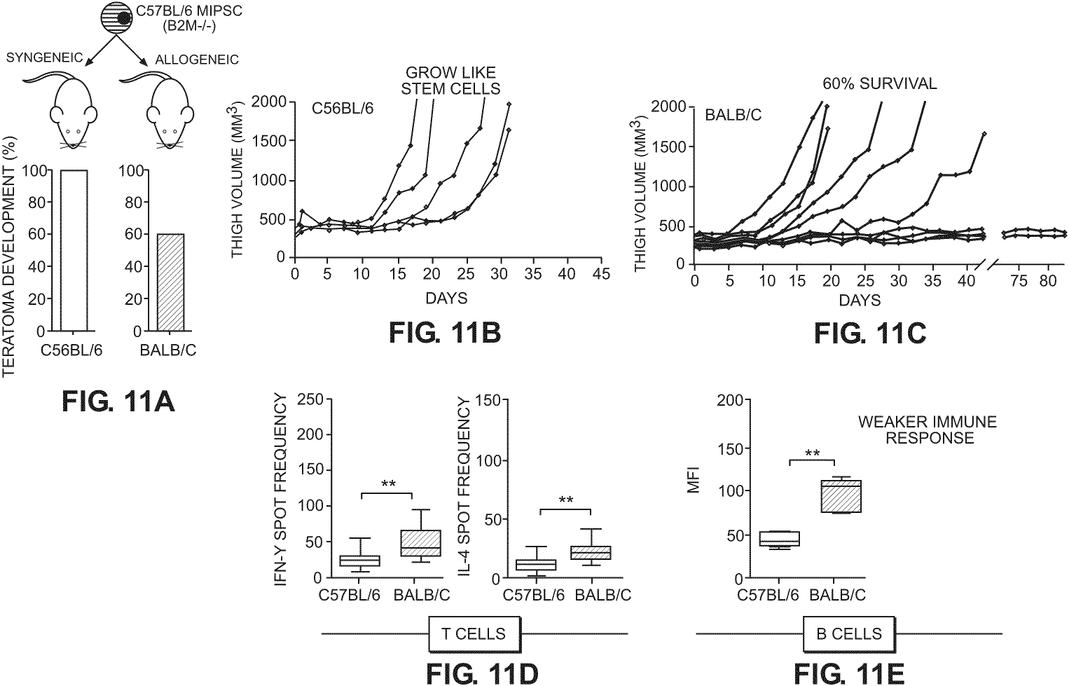

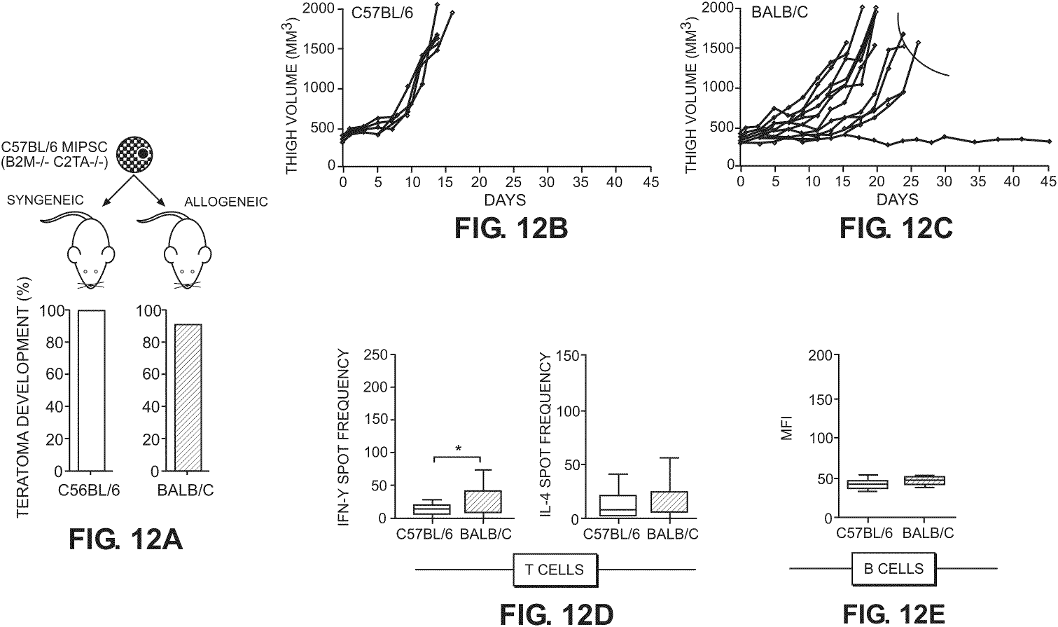

The present invention is directed to the exploitation of the fetomaternal tolerance that exists in pregnant women. Although half of a fetus’ human leukocyte antigens (HLA) are paternally inherited and the fetus expresses major HLA mismatched antigens, the maternal immune system does not recognize the fetus as an allogenic entity and does not initiate an immune response, e.g. as is seen in a “host versus graft” type of immune reaction. Fetomaternal tolerance is mainly mediated by syncytiotrophoblast cells in the fetal-maternal interface. As shown in FIG. 7, syncytiotrophoblast cells show little or no proteins of the major histocompatibility complexes I and II (MHC-I and MHC-II), as well as increased expression of CD47, known as the “don’t eat me” protein that suppresses phagocytic innate immune surveillance and elimination of HLA-devoid cells. Surprisingly, the same tolerogenic mechanisms that prevent rejection of the fetus during pregnancy also allow the HIP cells of the invention to escape rejection and facilitate long-term survival and engraftment of these cells after allogeneic transplantation.

These results are additionally surprising in that this fetomaternal tolerance can be introduced with as little as three genetic modifications (as compared to the starting iPSCs, e.g. hiPSCs), two reductions in activity (“knock outs” as further described herein) and one increase in activity (a “knock in” as described herein). Generally, others of skill in the art have attempted to suppress immunogenicity of iPSCs but have been only partially successful; see Rong et al., Cell Stem Cell 14:121-130 (2014) and Gomalusse et al., Nature Biotech doi:10.1038/nbt.3860).

Thus, the invention provides for the generation of HIP cells from pluripotent stem cells, and then their maintenance, differentiation and ultimately transplantation of their derivatives into patients in need thereof.

### B. Definitions

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.)

“Pluripotent stem cells” as used herein have the potential to differentiate into any of the three germ layers: endoderm (e.g. the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells, and “miPSCs” are murine induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. As described herein, cells do not need to pass through pluripotency to be reprogrammed into endodermal progenitor cells and/or hepatocytes.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “totipotent” means the ability of a cell to form an entire organism. For example, in mammals, only the zygote and the first cleavage stage blastomeres are totipotent.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells, the endodermal progenitor cells, and the hepatocytes can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

Cells can be from, for example, human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human mammal.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “hypo-immunogenic pluripotent cell” or “HIP cell” herein is meant a pluripotent cell that retains its pluripotent characteristics and yet gives rise to a reduced immunological rejection response when transferred into an allogeneic host. In preferred embodimements, HIP cells do not give rise to an immune response. Thus, “hypo-immunogenic” refers to a significantly reduced or eliminated immune response when compared to the immune response of a parental (i.e. “wt”) cell prior to immunoengineering as outlined herein. In many cases, the HIP cells are immunologically silent and yet retain pluripotent capabilities. Assays for HIP characteristics are outlined below.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

“β-2 microglobulin” or “β2M” or “B2M” protein refers to the human β2M protein that has the amino acid and nucleic acid sequences shown below; the human gene has accession number NC_000015.10:44711487-44718159.

“CD47 protein” protein refers to the human CD47 protein that has the amino acid and nucleic acid sequences shown below; the human gene has accession number NC_000016.10:10866208-10941562.

“CIITa protein” protein refers to the human CIITA protein that has the amino acid and nucleic acid sequences shown below; the human gene has accession number NC_000003.12:108043094-108094200.

By “wild type” in the context of a cell means a cell found in nature. However, in the context of a pluripotent stem cell, as used herein, it also means an iPSC that may contain nucleic acid changes resulting in pluripotency but did not undergo the gene editing procedures of the invention to achieve hypo-immunogenicity.

By “syngeneic” herein refers to the genetic similarity or identity of a host organism and a cellular transplant where there is immunological compatibility; e.g. no immune response is generated.

By “allogeneic” herein refers to the genetic dissimilarity of a host organism and a cellular transplant where an immune response is generated.

By “B2M-/-” herein is meant that a diploid cell has had the B2M gene inactivated in both chromosomes. As described herein, this can be done in a variety of ways.

By “CIITA -/-” herein is meant that a diploid cell has had the CIITA gene inactivated in both chromosomes. As described herein, this can be done in a variety of ways.

By “CD47 tg” (standing for “transgene”) or “CD47+”) herein is meant that the host cell expresses CD47, in some cases by having at least one additional copy of the CD47 gene.

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7, Oct-8, Oct-9, and Oct-11. Oct3/4 (referred to herein as “Oct4”) contains the POU domain, a 150 amino acid sequence conserved among Pit-1, Oct-1, Oct-2, and uric-86. (See, Ryan, A. K. & Rosenfeld, M. G., Genes Dev. 11:1207-1225 (1997), incorporated herein by reference in its entirety.) In some embodiments, variants have at least 85%, 90%, or 95% amino acid sequence identity across their whole sequence compared to a naturally occurring Oct polypeptide family member such as to those listed above or such as listed in Genbank accession number NP-002692.2 (human Oct4) or NP-038661.1 (mouse Oct4). Oct polypeptides (e.g., Oct3/4 or Oct 4) can be from human, mouse, rat, bovine, porcine, or other animals. Generally, the same species of protein will be used with the species of cells being manipulated. The Oct polypeptide(s) can be a pluripotency factor that can help induce multipotency in non-pluripotent cells.

A “Klf polypeptide” refers to any of the naturally-occurring members of the family of Kruppel-like factors (Klfs), zinc-finger proteins that contain amino acid sequences similar to those of the Drosophila embryonic pattern regulator Kruppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. (See, Dang, D. T., Pevsner, J. & Yang, V. W., Cell Biol. 32:1103-1121 (2000), incorporated by reference herein in its entirety.) Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPS cells in mice, and related genes Klf1 and Klf5 did as well, although with reduced efficiency. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Klf polypeptide family member such as to those listed above or such as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals. Generally, the same species of protein will be used with the species of cells being manipulated. The Klf polypeptide(s) can be a pluripotency factor. The expression of the Klf4 gene or polypeptide can help induce multipotency in a starting cell or a population of starting cells.

A “Myc polypeptide” refers to any of the naturally-occurring members of the Myc family. (See, e.g., Adhikary, S. & Eilers, M., Nat. Rev. Mol. Cell Biol. 6:635-645 (2005), incorporated by reference herein in its entirety.) It also includes variants that maintain similar transcription factor activity when compared to the closest related naturally occurring family member (i.e. within at least 50%, 80%, or 90% activity). It further includes polypeptides comprising at least the DNA-binding domain of a naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals. Generally, the same species of protein will be used with the species of cells being manipulated. The Myc polypeptide(s) can be a pluripotency factor.

A “Sox polypeptide” refers to any of the naturally-occurring members of the SRY-related HMG-box (Sox) transcription factors, characterized by the presence of the high-mobility group (HMG) domain, or variants thereof that maintain similar transcription factor activity when compared to the closest related naturally occurring family member (i.e. within at least 50%, 80%, or 90% activity). It also includes polypeptides comprising at least the DNA-binding domain of the naturally occurring family member, and can further comprise a transcriptional activation domain. (See, e.g., Dang, D. T. et al., Int. J. Biochem. Cell Biol. 32:1103-1121 (2000), incorporated by reference herein in its entirety.) Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals. Generally, the same species of protein will be used with the species of cells being manipulated. The Sox polypeptide(s) can be a pluripotency factor. As discussed herein, SOX2 proteins find particular use in the generation of iPSCs.

By “differentiated hypo-immunogenic pluripotent cells” or “differentiated HIP cells” or “dHIP cells” herein is meant iPS cells that have been engineered to possess hypoimmunogenicity (e.g. by the knock out of B2M and CIITA and the knock in of CD47) and then are differentiated into a cell type for ultimate transplantation into subjects. Thus, for example HIP cells can be differentiated into hepatocytes (“dHIP hepatocytes”), into beta-like pancreatic cells or islet organoids (“dHIP beta cells”), into endothelial cells (“dHIP endothelial cells”), etc.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat′l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” are agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” are agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at Ph 6.5 with 750 Mm sodium chloride, 75 Mm sodium citrate at 42° C.; or (3) overnight hybridization in a solution that employs 50% formamide, 5 × SSC (0.75 M NaCl, 0.075 M sodium citrate), 50 Mm sodium phosphate (Ph 6.8), 0.1 % sodium pyrophosphate, 5 × Denhardt’s solution, sonicated salmon sperm DNA (50 µl/ml), 0.1% SDS, and 10% dextran sulfate at 42° C., with a 10 minute wash at 42° C. in 0.2 × SSC (sodium chloride/sodium citrate) followed by a 10 minute high-stringency wash consisting of 0.1 × SSC containing EDTA at 55° C.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In one embodiment, an amino acid change in a CD47, HSVtk, EC-CD, or iCasp9 variant polypeptide prepared according to the methods described herein differentiates it from the corresponding parent that has not been modified according to the methods described herein, such as wild-type proteins, a naturally occurring mutant proteins or another engineered protein that does not include the modifications of such variant polypeptide. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell; e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells’ offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene. In general, knock in technologies result in the integration of the extra copy of the transgene into the host cell.

### VII. Cells of the Invention

The invention provides compositions and methodologies for generating HIP cells, starting with wild type cells, rendering them pluripotent (e.g. making induced pluripotent stem cells, or iPSCs), then generating HIP cells from the iPSC population.

### A. Methodologies for Genetic Alterations

The invention includes methods of modifying nucleic acid sequences within cells or in cell-free conditions to generate both pluripotent cells and HIP cells. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

As will be appreciated by those in the art, a number of different techniques can be used to engineer the pluripotent cells of the invention, as well as the engineering of the iPSCs to become hypo-immunogenic as outlined herein.

In general, these techniques can be used individually or in combination. For example, in the generation of the HIP cells, CRISPR may be used to reduce the expression of active B2M and/or CIITA protein in the engineered cells, with viral techniques (e.g. lentivirus) to knock in the CD47 functionality. Also, as will be appreciated by those in the art, although one embodiment sequentially utilizes a CRISPR step to knock out B2M, followed by a CRISPR step to knock out CIITA with a final step of a lentivirus to knock in the CD47 functionality, these genes can be manipulated in different orders using different technologies.

As is discussed more fully below, transient expression of reprogramming genes is generally done to generate/induce pluripotent stem cells.

### A. CRISPR Technologies

In one embodiment, the cells are manipulated using clustered regularly interspaced short palindromic repeats)/Cas (“CRISPR”) technologies as is known in the art. CRISPR can be used to generate the starting iPSCs or to generate the HIP cells from the iPSCs. There are a large number of techniques based on CRISPR, see for example Doudna and Charpentier, Science doi:10.1126/science.1258096, hereby incorporated by reference. CRISPR techniques and kits are sold commercially.

### B. TALEN Technologies

In some embodiments, the HIP cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

### C. Zinc Finger Technologies

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

### D. Viral Based Technologies

There are a wide variety of viral techniques that can be used to generate the HIP cells of the invention (as well as for the original generation of the iPCSs), including, but not limited to, the use of retroviral vectors, lentiviral vectors, adenovirus vectors and Sendai viral vectors. Episomal vectors used in the generation of iPSCs are described below.

### E. Down Regulation of Genes Using Interfering RNA

In other embodiments, genes that encode proteins used in HLA molecules are downregulated by RNAi technologies. RNA interference (RNAi) is a process where RNA molecules inhibit gene expression often by causing specific mRNA molecules to degrade. Two types of RNA molecules - microRNA (miRNA) and small interfering RNA (siRNA) -are central to RNA interference. They bind to the target mRNA molecules and either increase or decrease their activity. RNAi helps cells defend against parasitic nucleic acids such as those from viruses and transposons. RNAi also influences development.

sdRNA molecules are a class of asymmetric siRNAs comprising a guide (antisense) strand of 19-21 bases. They contain a 5′ phosphate, 2′Ome or 2′F modified pyrimidines, and six phosphotioates at the 3′ positions. They also contain a sense strand containing 3′ conjugated sterol moieties, 2 phospotioates at the 3′ position, and 2′Ome modified pyrimidines. Both strands contain 2′ Ome purines with continuous stretches of unmodified purines not exceeding a length of 3. sdRNA is disclosed in U.S. Pat. No. 8,796,443, incorporated herein by reference in its entirety.

For all of these technologies, well known recombinant techniques are used, to generate recombinant nucleic acids as outlined herein. In certain embodiments, the recombinant nucleic acids (either than encode a desired polypeptide, e.g. CD47, or disruption sequences) may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector’s copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s).

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

### B. Generation of Pluripotent Cells

The invention provides methods of producing non-immunogenic pluripotent cells from pluripotent cells. Thus, the first step is to provide the pluripotent stem cells.

The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7(1):116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POUSF1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

In general, as is known in the art, iPSCs are made from non-pluripotent cells such as CD34+ cord blood cells, fibroblasts, etc., by transiently expressing the reprogramming factors as described herein.

For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors for purposes of the invention may be via induction or modulation of an endogenous locus or from expression from a transgene.

For example, murine iPSCs can be generated using the methods of Diecke et al, Sci Rep. 2015, Jan. 28;5:8081 (doi: 10.1038/srep08081), hereby incorporated by reference in its entirety and specifically for the methods and reagents for the generation of the miPSCs. See also, e.g., Burridge et al., PLoS One, 2011 6(4):18293, hereby incorporated by reference in its entirety and specifically for the methods outlined therein.

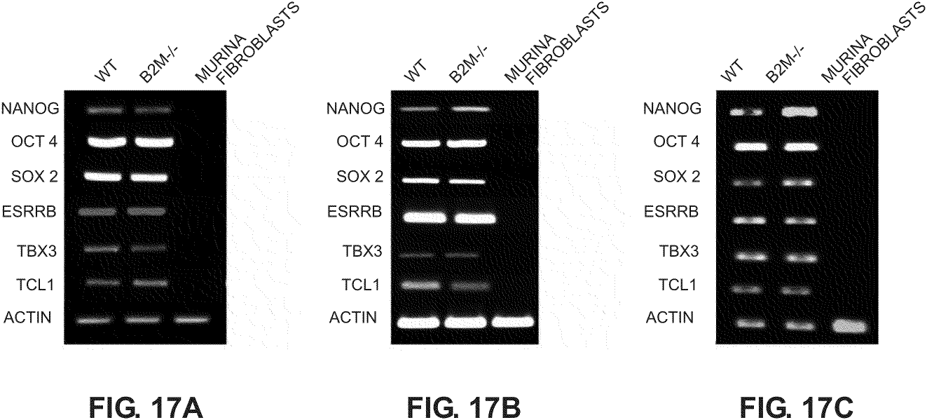

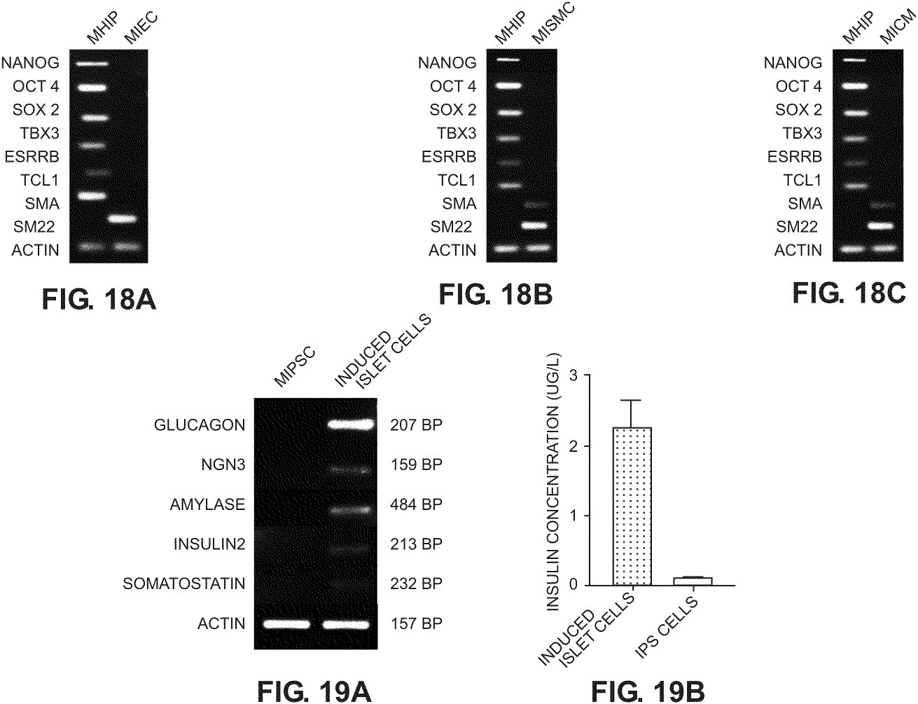

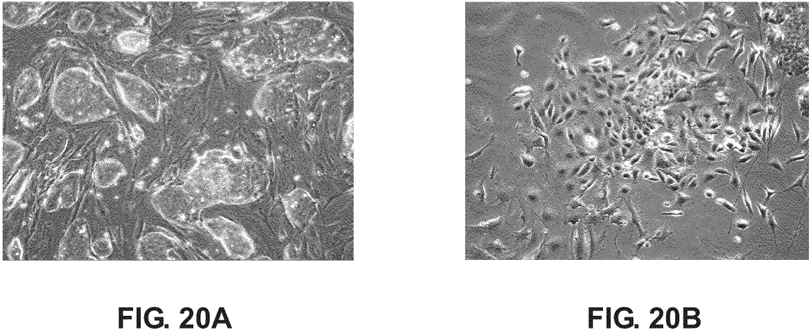

In some cases, the pluripotency of the cells is measured or confirmed as outlined herein, for example by assaying for reprogramming factors as is generally shown in FIGS. 17 or by conducting differentiation reactions as outlined herein and in the Examples.

### C. Generation of Hypo-Immunogenic Pluripotent Cells

The present invention is directed to the generation, manipulation, growth and transplantation of hypo-immunogenic cells into a patient as defined herein. The generation of HIP cells from pluripotent cells is done with as few as three genetic changes, resulting in minimal disruption of cellular activity but conferring immunosilencing to the cells.

As discussed herein, one embodiment utilizes a reduction or elimination in the protein activity of MHC I and II (HLA I and II when the cells are human). This can be done by altering genes encoding their component. In one embodiment, the coding region or regulatory sequences of the gene are disrupted using CRISPR. In another embodiment, gene translation is reduced using interfering RNA technologies. The third change is a change in a gene that regulates susceptibility to macrophage phagocytosis, such as CD47, and this is generally a “knock in” of a gene using viral technologies.

In some cases, where CRISPR is being used for the genetic modifications, hiPSC cells that contain a Cas9 construct that enable high efficiency editing of the cell line can be used; see, e.g., the Human Episomal Cas9 iPSC cell line, A33124, from Life Technologies.

### 1. HLA-I Reduction

The HIP cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A,B,C antibodies that bind to the the alpha chain of the human major histocompatibility HLA Class I antigens.

### A. B2m Alteration

In one embodiment, the reduction in HLA-I activity is done by disrupting the expression of the β-2 microglobulin gene in the pluripotent stem cell, the human sequence of which is disclosed herein. This alteration is generally referred to herein as a gene “knock out”, and in the HIP cells of the invention it is done on both alleles in the host cell. Generally the techniques to do both disruptions is the same.

A particularly useful embodiment uses CRISPR technology to disrupt the gene. In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2M gene in mouse or the B2M gene in human. After gene editing, the transfected iPSC cultures are dissociated to single cells. Single cells are expanded to full-size colonies and tested for CRISPR edit by screening for presence of aberrant sequence from the CRISPR cleavage site. Clones with deletions in both alleles are picked. Such clones did not express B2M/ B2M as demonstrated by PCR and did not express HLA-I as demonstrated by FACS analysis (see examples 1 and 6, for example).

Assays to test whether the B2M gene has been inactivated are known and described herein. In one embodiment, the assay is a Western blot of cells lysates probed with antibodies to the B2M protein. In another embodiment, reverse transcriptase polymerase chain reactions (rt-PCR) confirms the presence of the inactivating alteration.

In addition, the cells can be tested to confirm that the HLA I complex is not expressed on the cell surface. This may be assayed by FACS analysis using antibodies to one or more HLA cell surface components as discussed above.

It is noteworthy that others have had poor results when trying to silence the B2M genes at both alleles. See, e.g. Gornalusse et al., Nature Biotech. Doi/10.1038/nbt.3860).

### 2. HLA-II Reduction

In addition to a reduction in HLA I, the HIP cells of the invention also lack MHC II function (HLA II when the cells are derived from human cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II function (HLA II when the cells are derived from human cells) in the pluripotent cells or their derivatives can be measured using techniques known in the art such as Western blotting using antibodies to the protein, FACS techniques, rt-PCR techniques, etc.

### A. CIITA Alteration

In one embodiment, the reduction in HLA-II activity is done by disrupting the expression of the CIITA gene in the pluripotent stem cell, the human sequence of which is shown herein. This alteration is generally referred to herein as a gene “knock out”, and in the HIP cells of the invention it is done on both alleles in the host cell.

Assays to test whether the CIITA gene has been inactivated are known and described herein. In one embodiment, the assay is a Western blot of cells lysates probed with antibodies to the CIITA protein. In another embodiment, reverse transcriptase polymerase chain reactions (rt-PCR) confirms the presence of the inactivating alteration.

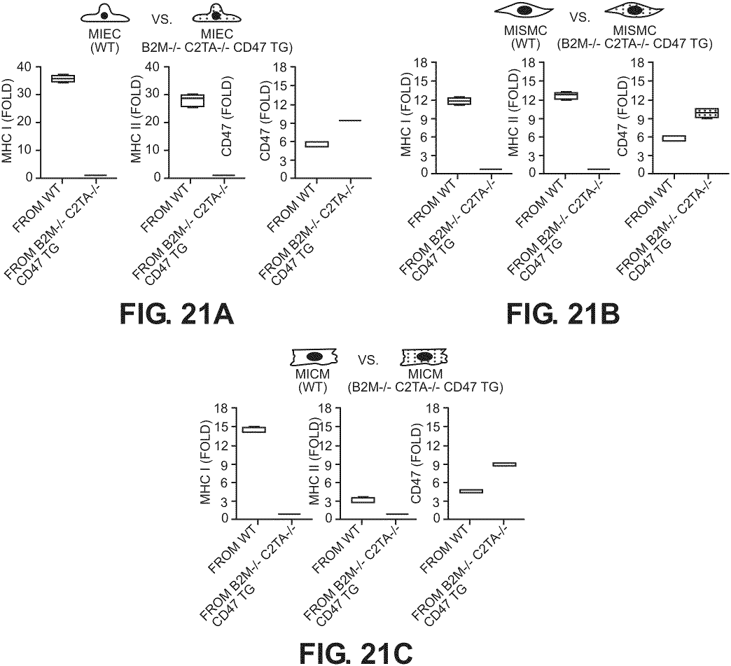

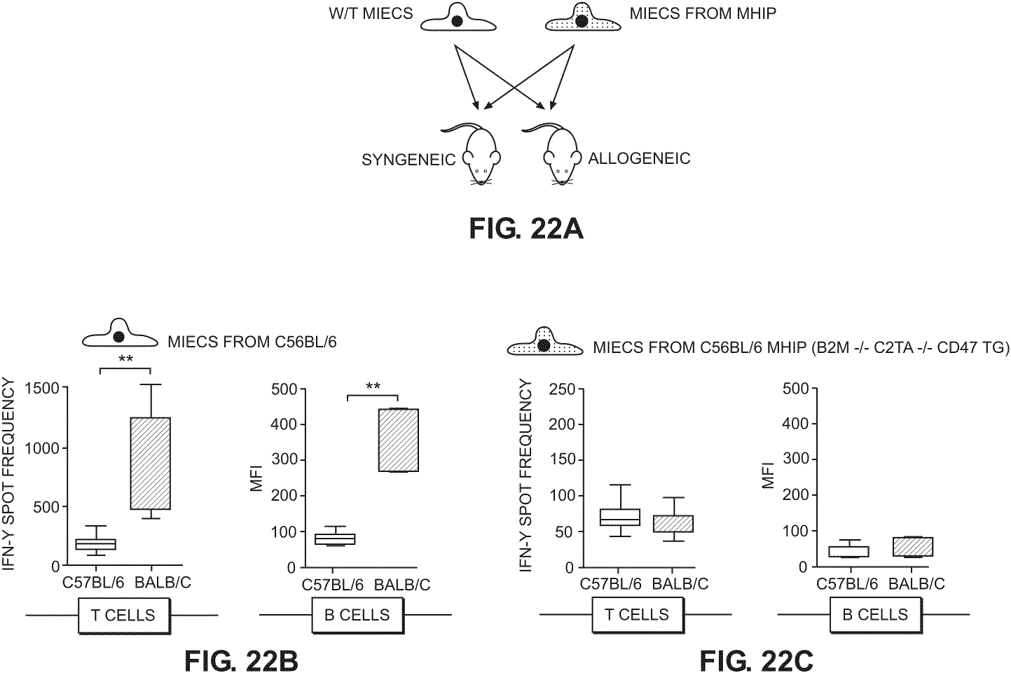

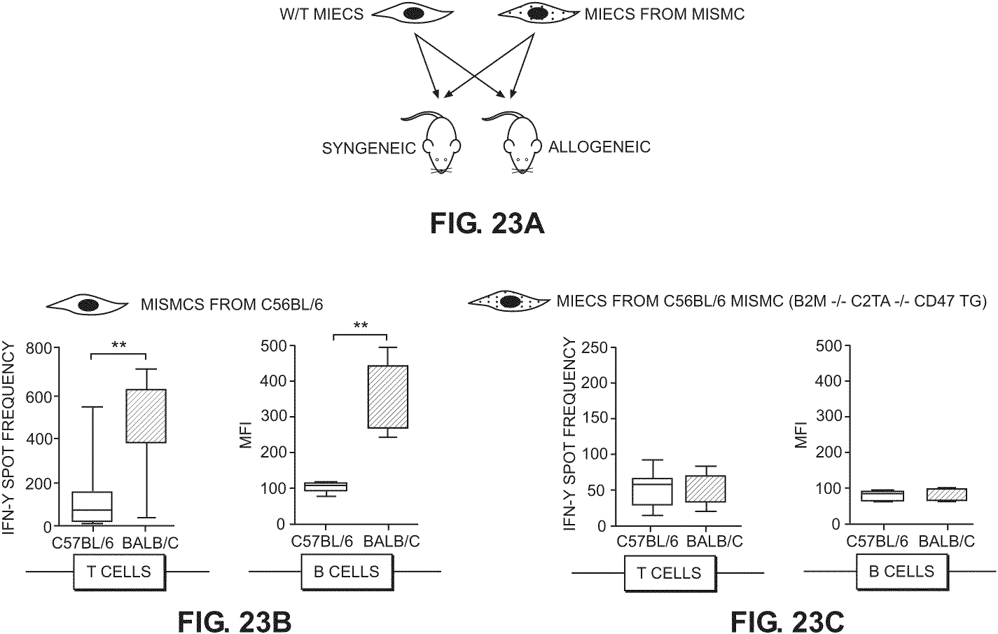

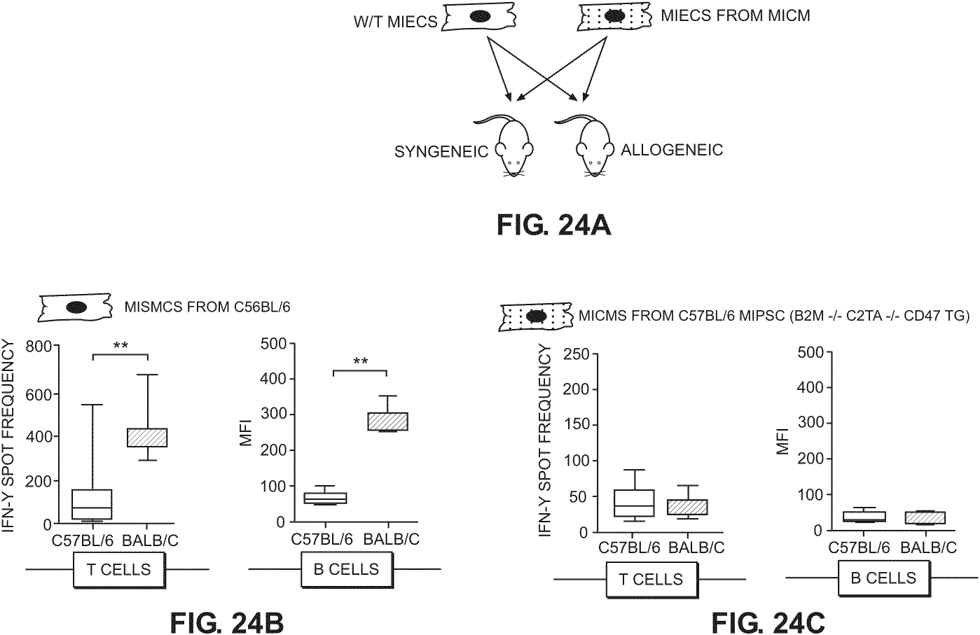

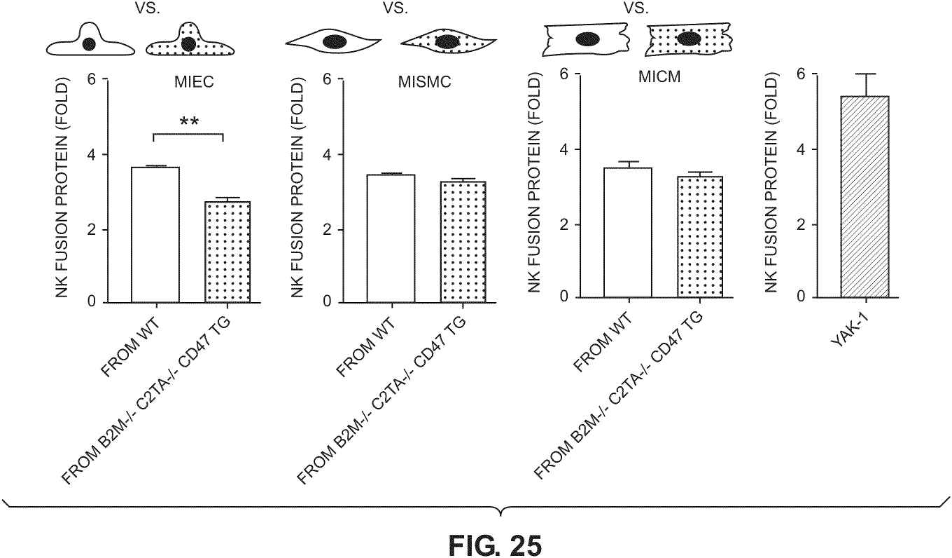

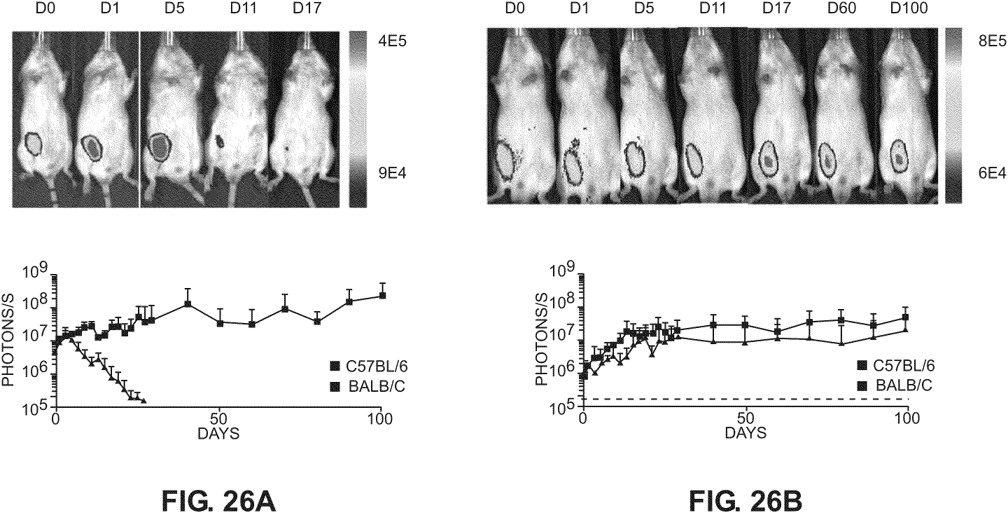

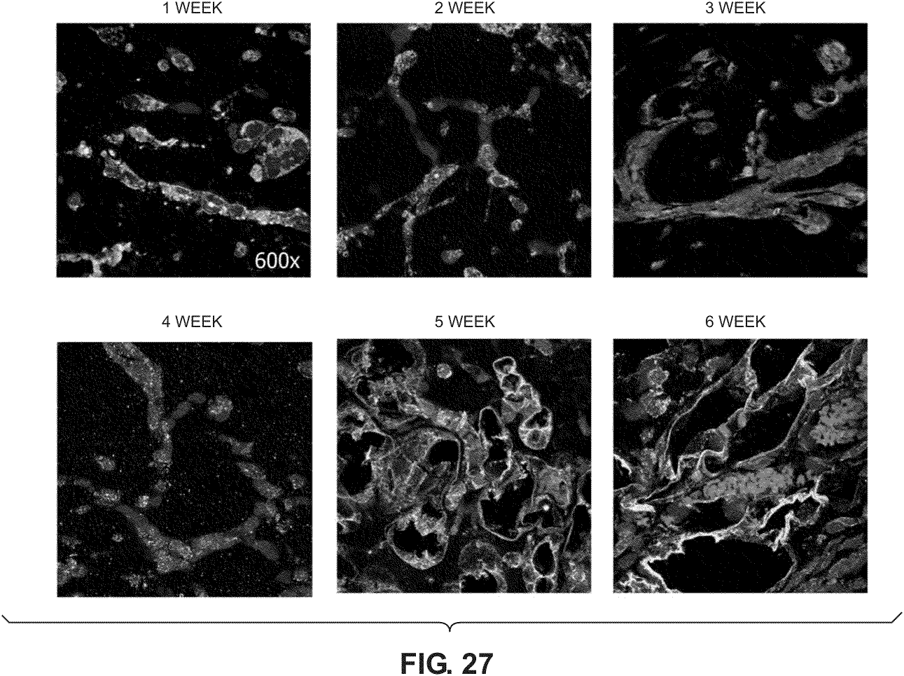

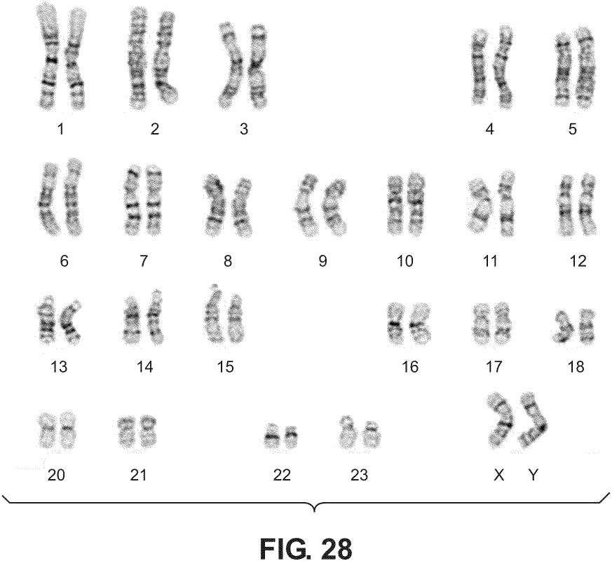

In addition, the cells can be tested to confirm that the HLA II complex is not expressed on the cell surface. Again, this assay is done as is known in the art (See FIGS. 21, for example) and generally is done using either Western Blots or FACS analysis based on commercial antibodies that bind to human HLA Class II HLA-DR, DP and most DQ antigens as outlined below.

A particularly useful embodiment uses CRISPR technology to disrupt the CIITA gene. CRISPRs were designed to target the coding sequence of the Ciita gene in mouse or the CIITA gene in human, an essential transcription factor for all MHC II molecules. After gene editing, the transfected iPSC cultures were dissociated into single cells. They were expanded to full-size colonies and tested for successful CRISPR editing by screening for the presence of an aberrant sequence from the CRISPR cleavage site. Clones with deletions did not express Ciita/ CIITA as determined by PCR and did not express MHC II/ HLA-II as determined by FACS analysis.

### 3. Phagocytosis Reduction

In addition to the reduction of HLA I and II (or MHC I and II), generally using B2M and CIITA knock-outs, the HIP cells of the invention have a reduced susceptibility to macrophage phagocytosis and NK cell killing. The resulting HIP cells “escape” the immune macrophage and innate pathways due toone or more CD47 transgenes.

### A. CD47 Increase

In some embodiments, reduced macrophage phagocytosis and NK cell killing susceptibility results from increased CD47 on the HIP cell surface. This is done in several ways as will be appreciated by those in the art using “knock in” or transgenic technologies. In some cases, increased CD47 expression results from one or more CD47 transgene.

Accordingly, in some embodiments, one or more copies of a CD47 gene is added to the HIP cells under control of an inducible or constitutive promoter, with the latter being preferred. In some embodiments, a lentiviral construct is employed as described herein or known in the art. CD47 genes may integrate into the genome of the host cell under the control of a suitable promoter as is known in the art.

The HIP cell lines were generated from B2M-/- CIITA-/- iPSCs. Cells containing lentivirus vectors expressing CD47 were selected using a Blasticidin marker. The CD47 gene sequence was synthesized and the DNA was cloned into the plasmid Lentivirus pLenti6/V5 with a blasticidin resistance (Thermo Fisher Scientific, Waltham, MA)

In some embodiments, the expression of the CD47 gene can be increased by altering the regulatory sequences of the endogenous CD47 gene, for example, by exchanging the endogenous promoter for a constitutive promoter or for a different inducible promoter. This can generally be done using known techniques such as CRISPR.

Once altered, the presence of sufficient CD47 expression can be assayed using known techniques such as those described in the Examples, such as Western blots, ELISA assays or FACS assays using anti-CD47 antibodies. In general, “sufficiency” in this context means an increase in the expression of CD47 on the HIP cell surface that silences NK cell killing. The natural expression levels on cells is too low to protect them from NK cell lysis once their MHC I is removed.

### 4. Suicide Genes

In some embodiments, the invention provides hypoimmunogenic pluripotent cells that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10):1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In one embodiment, the portion of the Caspase protein is exemplified in SEQ ID NO:6. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al, N. Engl. J. Med 365;18 (2011); Tey et al., Biol. Blood Marrow Transplant. 13:913-924 (2007), each of which are incorporated by reference herein in their entirety.)

### 5. Assays for HIP Phenotypes and Retention of Pluripotency

Once the HIP cells have been generated, they may be assayed for their hypo-immunogenicity and/or retention of pluripotency as is generally described herein and in the examples.

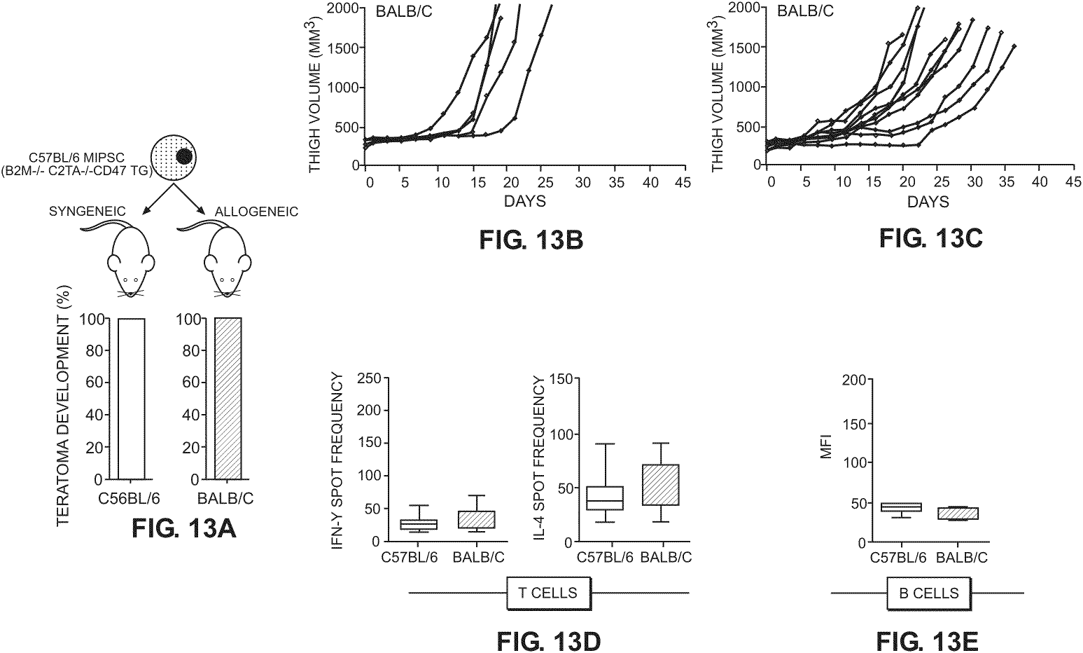

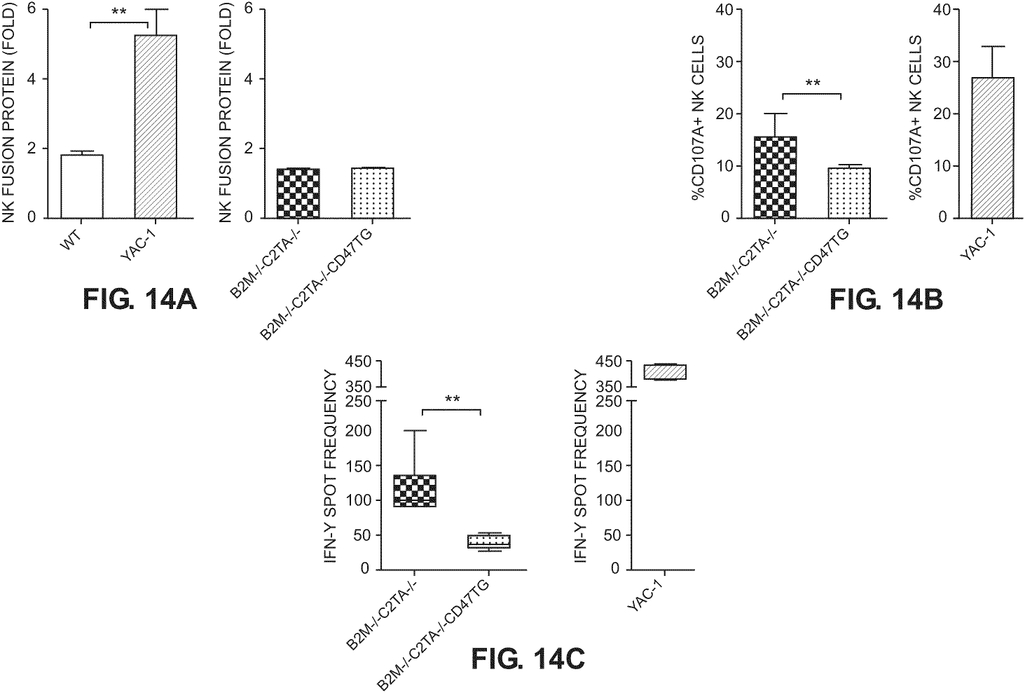

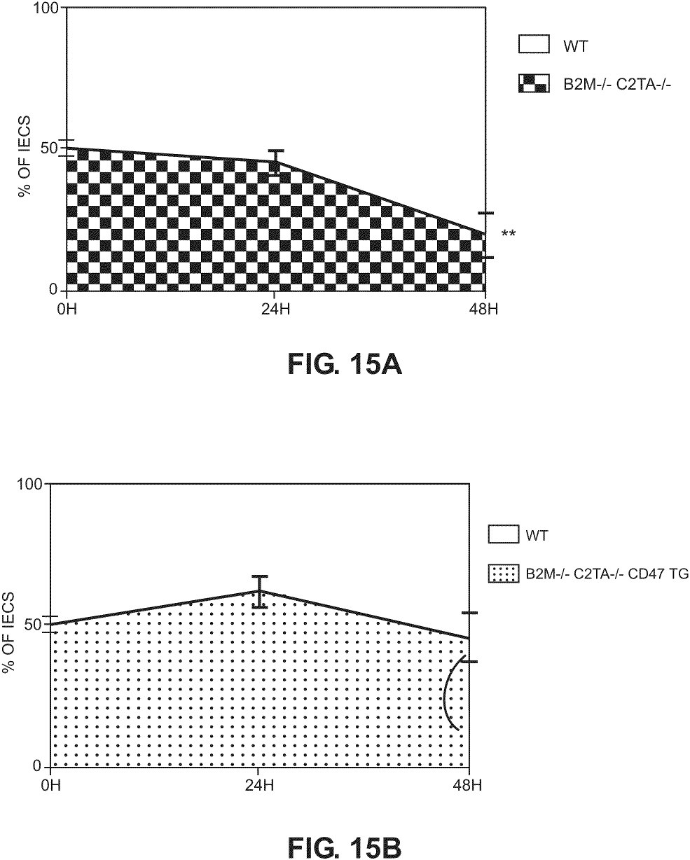

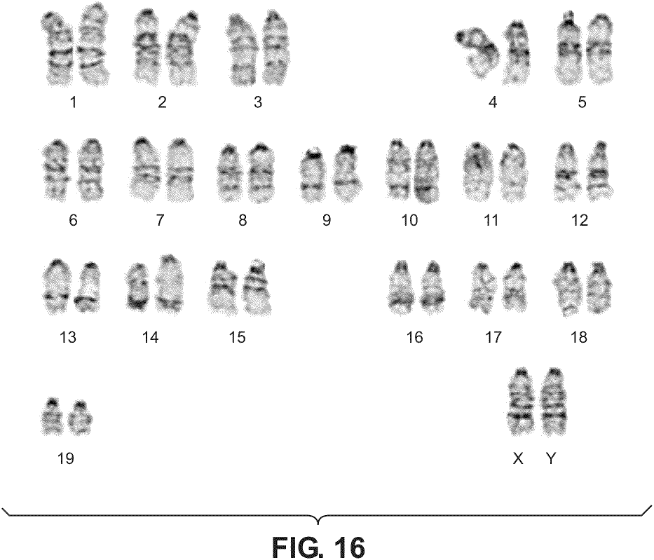

For example, hypo-immunogenicity are assayed using a number of techniques as exemplified in FIGS. 13 and FIGS. 15. These techniques include transplantation into allogeneic hosts and monitoring for HIP cell growth (e.g. teratomas) that escape the host immune system. HIP derivatives are transduced to express luciferase and can then followed using bioluminescence imaging. Similarly, the T cell and/or B cell response of the host animal to the HIP cells are tested to confirm that the HIP cells do not cause an immune reaction in the host animal. T cell function is assessed by Elispot, Elisa, FACS, PCR, or mass cytometry (CYTOF). B cell response or antibody response is assessed using FACS or luminex. Additionally or alternatively, the cells may be assayed for their ability to avoid innate immune responses, e.g. NK cell killing, as is generally shown in FIGS. 14. NK cell lytolytic activity is assessed in vitro or in vivo (as shown in FIGS. 15).

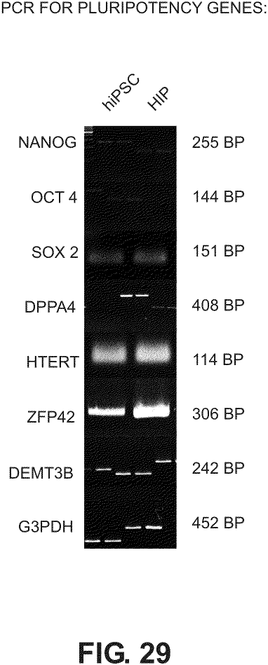

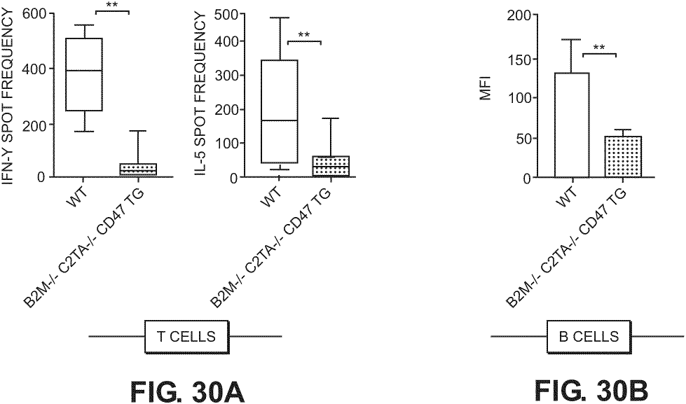

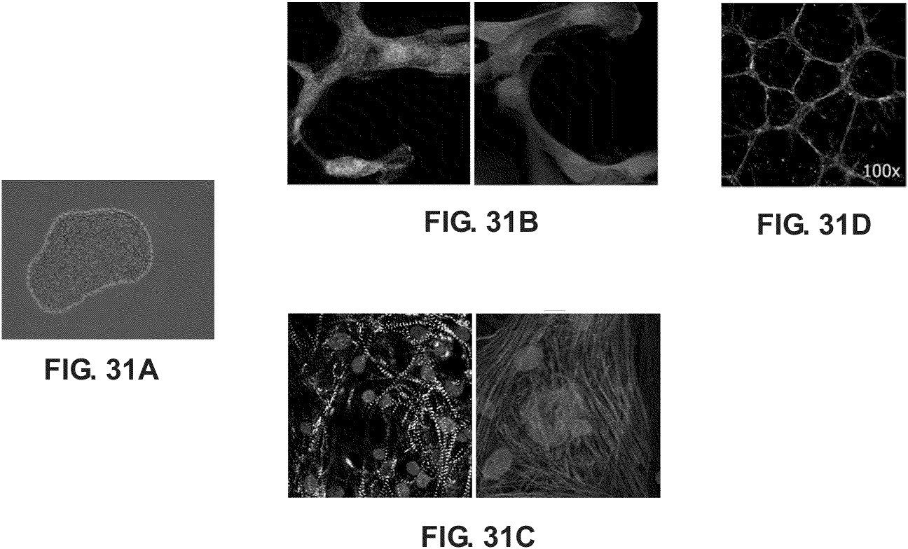

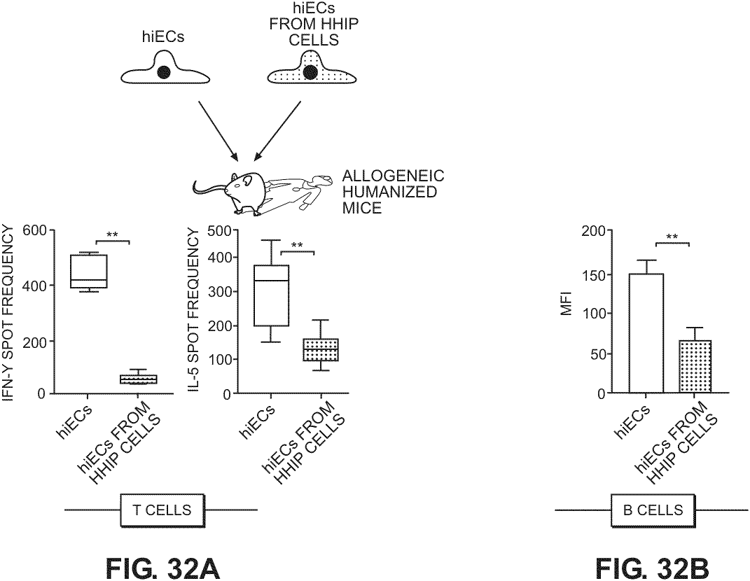

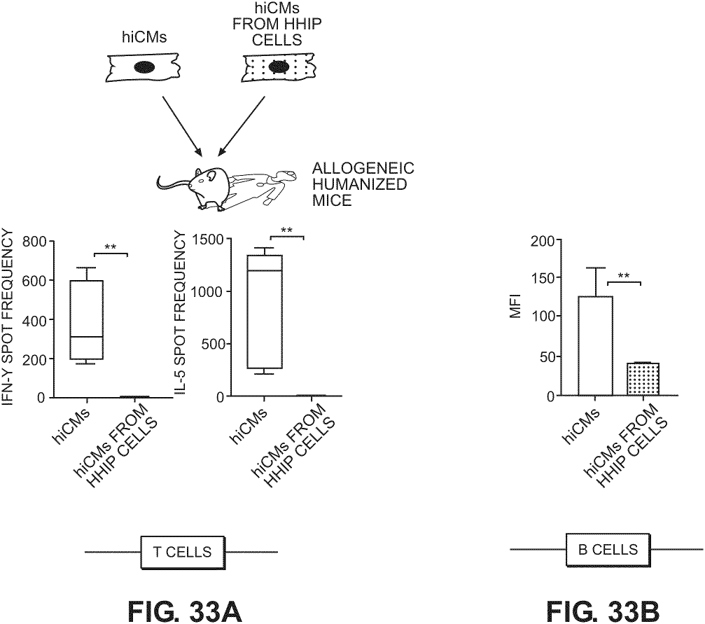

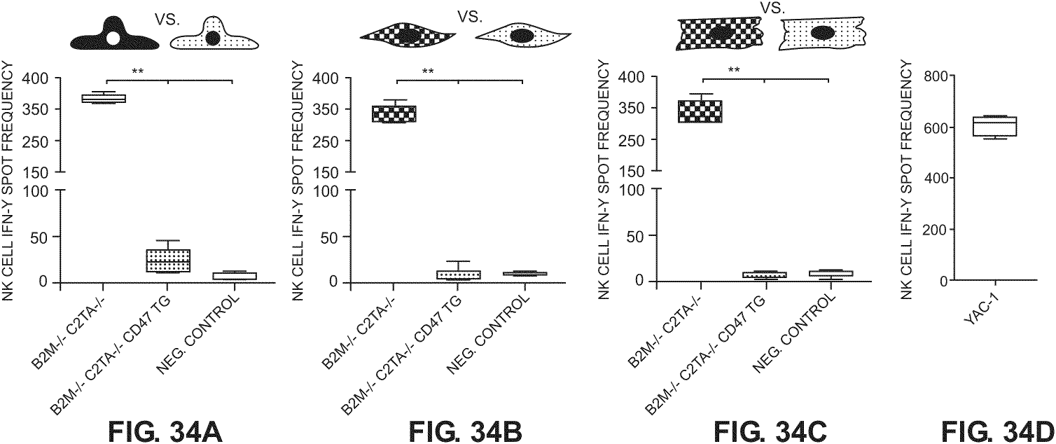

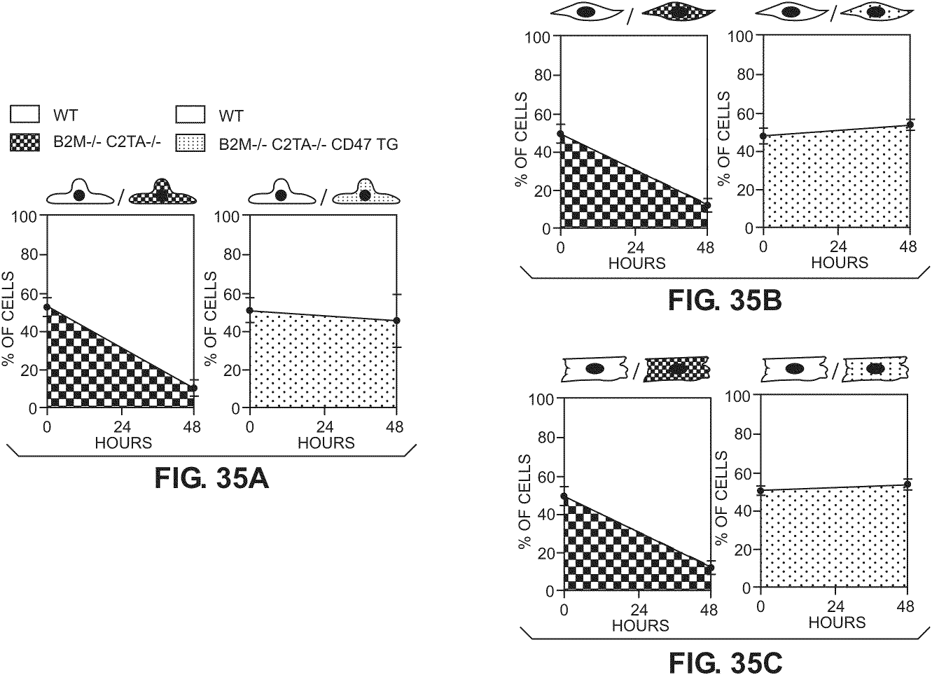

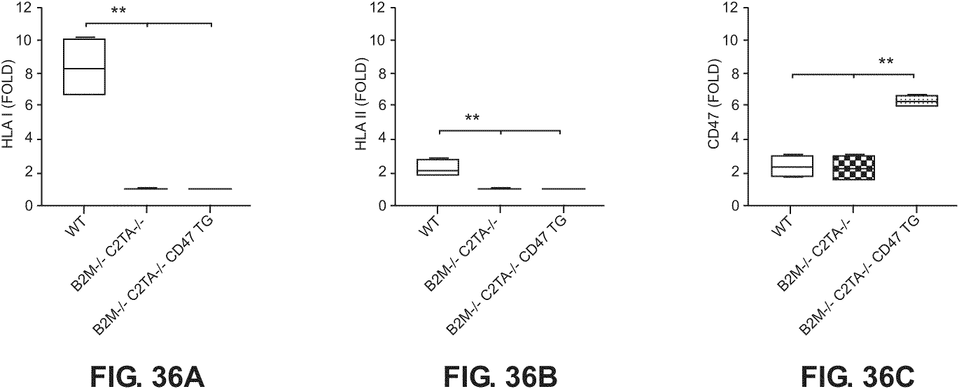

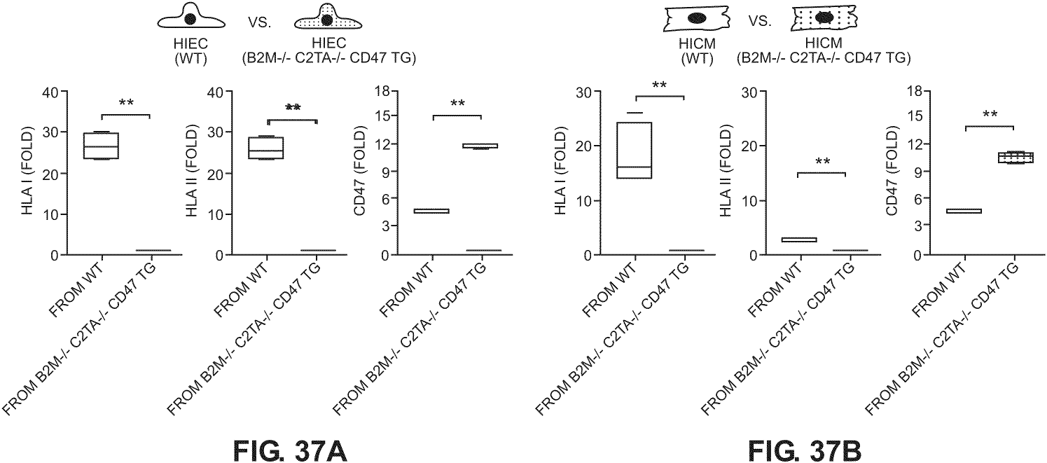

Similarly, the retention of pluripotency is tested in a number of ways. In one embodiment, pluripotency is assayed by the expression of certain pluripotency-specific factors as generally described herein and shown in FIG. 29. Additionally or alternatively, the HIP cells are differentiated into one or more cell types as an indication of pluripotency.

### D. Preferred Embodiments of the Invention

Provided herein are hypo-immunogenic pluripotent stem cells (“HIP cells”) that exhibit pluripotency but do not result in a host immune response when transplanted into an allogeneic host such as a human patient, either as the HIP cells or as the differentiated products of the HIP cells.

In one embodiment, human pluripotent stem cells (hiPSCs) are rendered hypo-immunogenic by a) the disruption of the B2M gene at each allele (e.g. B2M -/-), b) the disruption of the CIITA gene at each allele (e.g. CIITA -/-), and c) by the overexpression of the CD47 gene (CD47+, e.g. through introducing one or more additional copies of the CD47 gene or activating the genomic gene). This renders the hiPSC population B2M-/- CIITA -/-CD47tg. In a preferred embodiment, the cells are non-immunogenic. In another embodiment, the HIP cells are rendered non-immunogenic B2MCIIT Aas described above but are further modified by including an inducible suicide gene that is induced to kill the cells in vivo when required.

### E. Maintenance of HIP Cells

Once generated, the HIP cells can be maintained an undifferentiated state as is known for maintaining iPSCs. For example, HIP cells are cultured on Matrigel using culture media that prevents differentiation and maintains pluripotency.

### F. Differentiation of HIP Cells

The invention provides HIP cells that are differentiated into different cell types for subsequent transplantation into subjects. As will be appreciated by those in the art, the methods for differentiation depend on the desired cell type using known techniques. The cells are differentiated in suspension and then put into a gel matrix form, such as matrigel, gelatin, or fibrin/thrombin forms to facilitate cell survival. Differentiation is assayed as is known in the art, generally by evaluating the presence of cell-specific markers.

In some embodiments, the HIP cells are differentiated into hepatocytes to address loss of the hepatocyte functioning or cirrhosis of the liver. There are a number of techniques that can be used to differentiate HIP cells into hepatocytes; see for example Pettinato et al., doi:10.1038/spre32888, Snykers et al., Methods Mol Biol 698:305-314 (2011), Si-Tayeb et al, Hepatology 51:297-305 (2010) and Asgari et al., Stem Cell Rev (:493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the HIP cells are differentiated into beta-like cells or islet organoids for transplantation to address type I diabetes mellitus (T1DM). Cell systems are a promising way to address T1DM, see, e.g., Ellis et al., doi/10.1038/nrgastro.2017.93, incorporated herein by reference. Additionally, Pagliuca et al. reports on the successful differentiation of β-cells from hiPSCs (see doi/10.106/j.cell.2014.09.040, hereby incorporated by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; (doi:10.1038/nm.4030, hereby incorporated by reference in its entirety and in particular for the methods and reagents outlined there for the large-scale production of functional human β cells from human pluripotent stem cells).

Differentiation is assayed as is known in the art, generally by evaluating the presence of β cell associated or specific markers, including but not limited to, insulin. Differentiation can also be measured functionally, such as measuring glucose metabolism, see generally Muraro et al, doi:10.1016/j.cels.2016.09.002, hereby incorporated by reference in its entirety, and specifically for the biomarkers outlined there.

Once the dHIP beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

In some embodiments, the HIP cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi:10.1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See for example Kamao et al., doi:10.1016/j.stemcr.2013.12.007, hereby incorporated by reference in its entirety and specifically for the markers outlined in the first paragraph of the results section.

In some embodiments, the HIP cells are differentiated into cardiomyocytes to address cardiovascular diseases. Techniques are known in the art for the differentiation of hiPSCs to cardiomyoctes and discussed in the Examples. Differentiation can be assayed as is known in the art, generally by evaluating the presence of cardiomyocyte associated or specific markers or by measuring functionally; see for example Loh et al., doi:10.1016j. cell.2016.06.001, hereby incorporated by reference in its entirety and specifically for the methods of differentiating stem cells including cardiomyocytes.

In some embodiments, the HIP cells are differentiated into endothelial colony forming cells (ECFCs) to form new blood vessels to address peripheral arterial disease. Techniques to differentiate endothelial cells are known. See, e.g., Prasain et al., doi:10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the HIP cells are differentiated into thyroid progenitor cells and thyroid follicular organoids that can secrete thyroid hormones to address autoimmune thyroiditis. Techniques to differentiate thyroid cells are known the art. See, e.g. Kurmann et al., doi:10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

### G. Transplantation of Differentiated HIP Cells

As will be appreciated by those in the art, the differentiated HIP derivatives are transplated using techniques known in the art that depends on both the cell type and the ultimate use of these cells. In general, the dHIP cells of the invention are transplanted either intravenously or by injection at particular locations in the patient. When transplanted at particular locations, the cells may be suspended in a gel matrix to prevent dispersion while they take hold.

In order that the invention described herein may be more fully understood, the following examples are set forth. It should be understood that these examples are for illustrative purposes only and are not to be construed as limiting this invention in any manner.

### VIII. EXAMPLES

### A. General Techniques

### 1. Generation of Mouse iPSCs

These cells were generated using the methods of Diecke et al, Sci Rep. 2015, Jan. 28;5:8081 (doi:10.1038/srep08081), hereby incorporated in its entirety and specifically for the methods and reagents for the generation of the miPSCs.

Murine tail tip fibroblasts of mice were dissociated and isolated with collagenase type IV (Life Technologies, Grand Island, NY, USA) and maintained with Dulbecco’s modified Eagle medium (DMEM) containing 10% fetal bovine serum (FBS), L-glutamine, 4.5 g/L glucose, 100 U/mL penicillin, and 100 µg/mL streptomycin at 37° C., 20% O₂, and 5% CO₂ in a humidified incubator. 1×10⁶ murine fibroblasts were then reprogrammed using a novel codon optimized mini-intronic plasmid (co-MIP) (10-12 µm of DNA) expressing the four reprogramming factors Oct4, KLF4, Sox2 and c-Myc using the Neon Transfection system. After transfection, fibroblast were plated on a MEF feeder layer and kept in fibroblast media with the addition of sodium butyrate (0.2 mM) and 50 µg/mL ascorbic acid. When ESC-like colonies appeared, media was changed to murine iPSC media containing DMEM, 20% FBS, L-glutamine, non-essential amino acids (NEAA), β-mercaptoethanol, and 10 ng/mL leukemia inhibitory factor (LIF). After 2 passages, the murine iPSCs were transferred to 0.2% gelatin coated plates and further expanded. With every passage, the iPSCs were sorted for the murine pluripotency marker SSEA-1 using magnetic activated cell sorting (MACS).

### 2. Generation of Human iPSCs

The generation of hiPSCs was done as generally outlined in Burridge et al., PLoS One, 2011 6(4):18293, hereby incorporated by reference in its entirety and specifically for the methods outlined therein.

The Gibco® Human Episomal iPSC Line (Catalog No. A33124, Thermo Fisher Scientific) was derived from CD34+ cord blood using a three-plasmid, seven-factor (SOKMNLT; SOX2, OCT4 (POU5F1), KLF4, MYC, NANOG, LIN28, and SV40L T antigen) EBNA-based episomal system. This iPSC line is considered to be zero foot-print as there was no integration into the genome from the reprogramming event. It has been shown to be free of all reprogramming genes.

The Gibco® Human Episomal iPSC Line has a normal karyotype and endogenous expression of pluripotent markers like Oct4, Sox2, and Nanog (as shown by RT-PCR) and Oct4, SSEA4, TRA-1-60 and TRA-1-81 (as shown by ICC). Whole genome expression and epigenetic profiling analyses demonstrated that this episomal hiPSC line is molecularly indistinguishable from human embryonic stem cell lines (Burridge et al., 2011). In directed differentiation and teratoma analyses, these hiPSCs retained their differentiation potential for the ectodermal, endodermal, and mesodermal lineages (Burridge et al., 2011). In addition, vascular, hematopoietic, neural, and cardiac lineages were derived with robust efficiencies (Burridge et al., 2011).

### 3. FACS Analysis of Surface Molecules

### A. Detection of Human HLA I Surface Molecules

Human iPSCs, iCMs and iECs were plated in 6-well plates and stimulated with 100 ng/ml human IFNg (Peprotech, Rocket Hill, NJ). Cells were harvested and labeled with APC-conjugated HLA-A,B,C antibody (clone G46_2.6, cat. No.562006, BD BioSciences, San Jose, CA) or APC-conjugated IgG1 isotype control antibody (clone MOPC-21, cat. No. 555751, BD BioSciences). HLA-A,B,C antibody binds specific to the alpha chain of the human major histocompatibility HLA Class I antigens. Data analyzing was performed by Flow Cytometry (BD Bioscience) and results were expressed as fold change to isotype control.

### 4. Detection of Human HLA II Surface Molecules

Human iPSCs, iCMs and iECs were plated in 6-well plates and stimulated with 100 ng/ml human TNFa (Peprotech, Rocket Hill, NJ). Cells were harvested and labeled with Alexa-flour647-labeled HLA-DR,DP,DQ antibody (clone Tu3a, cat. No. 563591, BD BioSciences, San Jose, CA) or Alexa-flour647-labeled IgG2a isotype control antibody (clone G155-178, cat. No.557715, BD BioSciences). HLA- DR,DP,DQ antibody binds specific to human HLA Class II HLA-DR, DP and most DQ antigens. Data analyzing was performed by Flow Cytometry (BD Bioscience) and results were expressed as fold change to isotype control.

### 5. Detection of Human CD47 Surface Molecules

Human iPSCs, iCMs and iECs were plated in 6-well plates and stimulated with 100 ng/ml human IFNg (Peprotech, Rocket Hill, NJ). Cells were harvested and labeled with PerCP-Cy5-conjugated CD47 (clone B6H12, cat. No. 561261, BD BioSciences, San Jose, CA) or PerCP-Cy5-conjugated IgG1 isotype control antibody (clone MOPC-21, cat. No. 550795, BD BioSciences). The B6H12 CD47 monoclonal antibody specifically binds to CD47, a 42-52 kDa N-linked glycan protein. Data analyzing was performed by Flow Cytometry (BD Bioscience) and results were expressed as fold change to isotype control.

### 6. Detection of Mouse MHC I Surface Molecules

For the detection of MHC I surface molecules on miPSC, miEC, miSMC and miCM, cells were plated on gelatin coated 6-well plates and were stimulated with 100 ng/ml mouse IFNg (Peprotech, Rocket Hill, NJ). After harvesting, cells were labeled with PerCP-eFlour710-labeled MHCI antibody (clone AF6-88.5.5.3, cat.No. 46-5958-82, eBioscience, Santa Clara, CA) or PerCP-eFlour710-labeled IgG2b isotype control antibody. (clone eB149/10H5, cat.No. 46-4031-80, eBioscience). The MHCI antibody reacts with the H-2Kb MHC class I alloantigen. Data analyzing was performed by Flow Cytometry (BD Bioscience) and results were expressed as fold change to isotype control.

### 7. Detection of Mouse MHC II Surface Molecules

For the detection of MHC II surface molecules on miPSC, miEC, miSMC and miCM, cells were plated on gelatin coated 6-well plates and were stimulated with 100 ng/ml mouse TNFa (Peprotech, Rocket Hill, NJ). After harvesting, cells were labeled with PerCP-eFlour710-labeled MHC II antibody (clone M5/114.15.2, cat.No. 46-5321-82, eBioscience, Santa Clara, CA) or PerCP-eFlour710-labeled IgG2a/K isotype control antibody. (clone eBM2a, cat.No. 46-4724-80, eBioscience). The MHC II antibody reacts with the mouse major histocompatibility complex class II, both I-A and I-E subregion-encoded glycoproteins. Data analyzing was performed by Flow Cytometry (BD Bioscience) and results were expressed as fold change to isotype control.

### 8. Detection of Mouse Cd47 Surface Molecules

For the detection of Cd47 surface molecules on miPSC, miEC, miSMC and miCM, cells were plated on gelatin coated 6-well plates and were stimulated with 100 ng/ml mouse IFNg (Peprotech, Rocket Hill, NJ). After harvesting, cells were labeled with Alexa Fluor 647-labeled Cd47 antibody (clone miap301, cat.No. 563584, BD BioSciences, San Jose, CA) or Alexa Fluor 647-labeled IgG2a/K isotype control antibody. (clone R35-95, cat.No. 557690, BD BioSciences). The Cd47 antibody specifically binds to the extracellular domain of CD47, also known as Integrin-Associated Protein (IAP). Data analyzing was performed by Flow Cytometry (BD Bioscience) and results were expressed as fold change to isotype control.

### 9. Determining Mouse Cell Morphology In Vivo After Allogeneic Transplantation

Allogeneic mice were placed in an induction chamber and anaesthesia was induced with 2% isoflurane (Isothesia, Butler Schein). 1 mio cells, either miPSC-derived cardiomyocytes (miCM), miPSC-derived smooth muscle cells (miSMC) or miPSC-derived endothelial cells (miEC) in 250 ul 0.9% saline were mixed with 250 ul BD Matrigel High Concentration (1:1; BD Biosciences) and injected subcutaneously in the lower dorsa of mice using a 23-G syringe. Matrigel plugs were explantated 1, 2, 3, 4, 5, 6, 8, 10 and 12 weeks after implantation and were fixed with 4% paraformaldehyde and 1% Glutenaldehyde for 24h, followed by dehydration and embedding in paraffin. Section of 5 µm thickness were cut and stained with Hematoxylin and Eosin (HE).

### 10. Determining Human Cell Morphology In Vivo After Allogeneic Transplantation

Humanized NSG-SGM3 mice were placed in an induction chamber and anaesthesia was induced with 2% isoflurane (Isothesia, Butler Schein). 1 mio cells, either hiPSC-derived cardiomyocytes (hiCM) or hiPSC-derived endothelial cells (hiEC) in 250 ul 0.9% saline containing ZVAD (100 mM, benzyloxycarbonyl-Val-Ala-Asp(O-methyl)-fluoromethyl ketone, Calbiochem), Bcl-XL BH4 (cell-permeant TAT peptide, 50 nM, Calbiochem), cyclosporine A (200 nM, Sigma), IGF-1 (100 ng/ml, Peprotech) and pinacidil (50 mM, Sigma) were mixed with 250 ul BD Matrigel High Concentration (1:1; BD Biosciences) and injected subcutaneously in the lower dorsa of mice using a 23-G syringe. Matrigel plugs were explanted 2, 4, 6, 8, 10 and 12 weeks after implantation and were fixed with 4% paraformaldehyde and 1% Glutenaldehyde for 24 h, followed by dehydration and embedding in paraffin. Section of 5 µm thickness were cut and stained with Hematoxylin and Eosin (HE).

### B. Example 1: Generation of ß-2 Microglobulin Knockout Pluripotent Cells in Mouse Model

Induced Pluripotent Cell Generation: Hypoimmune pluripotent cells were generated in a mouse embodiment. Human hypoimmune pluripotent cells are another embodiment that are generated using the strategies described herein.

Mouse induced pluripotent stem cells (miPSCs) were generated from C57BL/6 fibroblasts. Mitomycin-inhibited CF1 Mouse embryonic fibroblast (MEF, Applied Stemcell, CA) were thawed and maintained in DMEM + GlutaMax 31966 (Gibco, Grand Island, NY) with 10% fetal calf sera heat inactivated (FCS hi), 1% MEM-NEAA and 1% Pen Strep (Thermo Fisher Scientific-Gibco, Waltham, MA). After the MEF feeder cells formed a 100% confluent monolayer, miPSCs were grown on MEF in KO DMEM 10829 with 15% KO Serum Replacement, 1% MEM-NEAA, 1% Pen Strep (Thermo Fisher-Gibco), 1× beta-mercaptoethanol and 100 units LIF (Millipore, Billerica, MA). Cells were maintained in 10 cm dishes, medium was changed daily and the cells were passaged every 2-3 days using 0.05% Trypsin-EDTA (Thermo Fisher-Gibco). miPSCs were cultured on gelatin (Millipore) without feeders using standard media. Cell cultures were regularly screened for mycoplasma infections using the MycoAlert Kit (Lonza, Cologne, Germany).

Mice: BALB/c (BALB/cAnNCrl, H2d), C57BL/6 (C57BL/6J, B6, H2b), BALB/c nude (BALB/c NU/NU, CAnN.CgFoxnl<nu>/Crl, H2d) and Scid beige (CBySmn.CB17-Prkdcscid/J) (all 6-12 weeks) were used as recipients for different assays (all 6-12 weeks of age). Mice were purchased from Charles River Laboratories (Sulzfeld, Germany) and received humane care in compliance with the Guide for the Principles of Laboratory Animals. Animal experiments were approved by the Hamburg “Amt für Gesundheit und Verbraucherschutz” and performed according to local and EU guidelines.

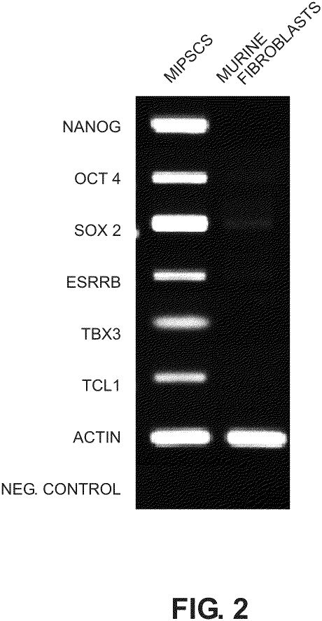

Pluripotency Confirmation: Pluripotency was shown by rtPCR. RNA was extracted using the PureLink RNA Mini Kit (Thermo Fisher Scientific). A DNase I step was included to remove contaminating genomic DNA. cDNA was generated using Applied Biosystems® High-Capacity cDNA Reverse Transcription Kit. No-reverse transcriptase (no-RT) controls were also generated from all RNA samples. Gene-specific primers were used to amplify target sequences using AmpliTaq Gold 360 Master Mix (Thermo Fisher Scientific-Applied Biosystems, Waltham, MA). PCR reactions were visualized on 2% agarose gels. A positive control primer set that amplifies a constitutively expressed housekeeping gene (Actb) that encodes a cellular cytoskeleton protein was included. Results are shown in FIG. 2. Pluripotency markers Nanog, Oct4, Sox2, Esrrb, Tbx3, Tcl1 were detected by rtPCR of miPSC cells but not the parental fibroblasts.

Pluripotency was also tested by immunofluorescence. miPSC were plated in 24-well dishes and processed for RT-PCR and immunocytochemistry (ICC) analysis 48 h after plating. For ICC, cells were fixed, permeabilized and blocked using the Image-iT Fixation/Permeabilization Kit (Thermo Fisher Scientific, Waltham, MA). Cells were stained overnight at 4° C. with primary antibodies for Sox2 and Oct4. After several washes the cells were incubated with an AlexaFluor 488 secondary antibody and NucBlue Fixed Cell ReadyProbes Reagent (Thermo Fisher Scientific). Stained cells were imaged using a fluorescent microscope and were positive for Sox2 and Oct4. Data not shown.

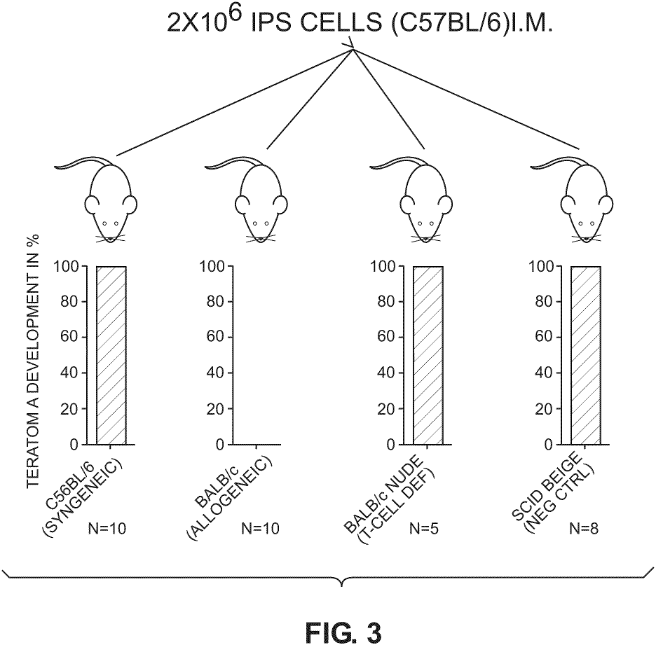

FIG. 3 shows further confirms pluripotency by a functional assay. 2 × 10⁶ miPSC cells were injected into the thigh muscle of recipient C57BL/6 (syngeneic), BALB/c (allogeneic), BALB/c nude (allogeneic but T-cell deficient), and scid beige (immunodeficient) mice. Teratomas were formed in all mice except the immunocompetent allogenic BALB/c mice.

ß-2 Microglobulin Knockout: CRISPR technology was used for the knockout of the B2m gene. For targeting the coding sequence of mouse β-2-microglobuline (B2m) gene, the CRISPR sequence 5′-TTCGGCTTCCCATTCTCCGG(TGG)-3′ was annealed and ligated into the All-In-One (AIO) vectors containing the Cas9 expression cassette as per the kit’s instructions (GeneArt CRISPR Nuclease Vector Kit, Thermo Fischer Scientific, Waltham, MA). (Another CRISPRs that worked but were less effective were 5′-GTATACTCACGCCACCCAC(CGG)-3′ and 5′-GGCGTATGTATCAGTCTCAG(TGG)-3′). miPSC were transfected with the AIO vectors using Neon electroporation with two 1200 V pulses of 20 ms duration. The transfected iPSC cultures were dissociated into single cells using 0.05% Trypsin (Gibco) and then sorted with FACSAria™ cell sorter (BD Bioscience, Franklin Lakes, NJ) for removing doublets and debris by selective gating on forward and side scatter emission. Single cells were expanded to full-size colonies and tested for the CRISPR edits by screening for the presence of the aberrant sequence from the CRISPR cleavage site. Briefly, the target sequence was amplified via PCR using AmpliTaq Gold Mastermix (Thermo Fisher-Applied Biosystems, Waltham, MA) and the primers B2m gDNA:

F: 5'-CTGGATCAGACATATGTGTTGGGA
-3',

R: 5'-GCAAAGCAGTTTTAAGTCCACACAG
-3'

After cleanup of the obtained PCR product (PureLink® Pro 96 PCR Purification Kit, Thermo Fisher Scientific, Waltham, MA), Sanger sequencing was performed using an Ion Personal Genome Machine (PGM™, Thermo Fisher Scientific). Sequencing for the identification of the homogeneity, a 250 bp region of the B2m gene, was PCR amplified using primers B2m gDNA PGM:

F: 5'-TTTTCAAAATGTGGGTAGACTTTGG-
3' and

R: 5'- GGATTTCAATGTGAGGCGGGT
-3'.

The PCR product was purified as previously described and prepared using the Ion PGM Hi-Q Template Kit (Thermo Fisher Scientific). Experiments were performed on the Ion PGM™ System with the Ion 318™ Chip Kit v2 (Thermo Fisher Scientific). Analysis for Pluripotency were performed again.

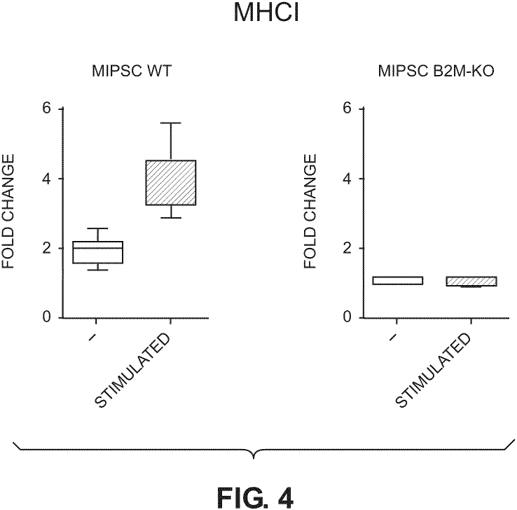

As seen in FIG. 4, ß-2-microglobulin expression was knocked out in the miPSC cells. MHC-I expression was not induced by IFN-γ stimulation (right panel). As a control, the parent miPSC cells were stimulated with IFN-γ (left panel).

### C. Example 2: Generation of ß-2 Microglobulin/Ciita Double-Knockout Pluripotent Cells

CRIPSR technology was used for the additional knockout of Ciita gene. For targeting the coding sequence of mouse Ciita gene, the CRISPR sequence 5′-GGTCCATCTGGTCATAGAGG (CGG)-3′ was annealed and ligated into the All-In-One (AIO) vectors containing the Cas9 expression cassette as per the kit’s instructions (GeneArt CRISPR Nuclease Vector Kit, Thermo Fischer, Waltham, MA). miPSC were transfected with the AIO vectors using the same condition for B2m-KO. The transfected iPSC cultures were dissociated into single cells using 0.05% Trypsin (Thermo Fisher-Gibco) and then sorted with FACSAria™ cell sorter (BD Bioscience, Franklin Lakes, NJ) for removing doublets and debris by selective gating on forward and side scatter emission. Single cells were expanded to full-size colonies and tested for CRISPR edits by screening for the presence of the aberrant sequence from the CRISPR cleavage site. Briefly, the target sequence was amplified via PCR using AmpliTaq Gold Mastermix (Thermo Fisher Applied Biosystems, Darmstadt, Germany) and the primers Ciita gDNA F: 5′-CCCCCAGAACGATGAGCTT-3′, R: 5′-TGCAGAAGTCCTGAGAAGGCC-3′. After cleanup of the obtained PCR product (PureLink® Pro 96 PCR Purification Kit, Thermo Fisher, Waltham, MA), Sanger sequencing was performed. Using the DNA sequence chromatogram, edited clones were then identified through the presence of the aberrant sequence from the CRISPR cleavage site. Indel size was calculated using the TIDE tool. PCR and ICC were performed again to verify the pluripotency status of the cells.

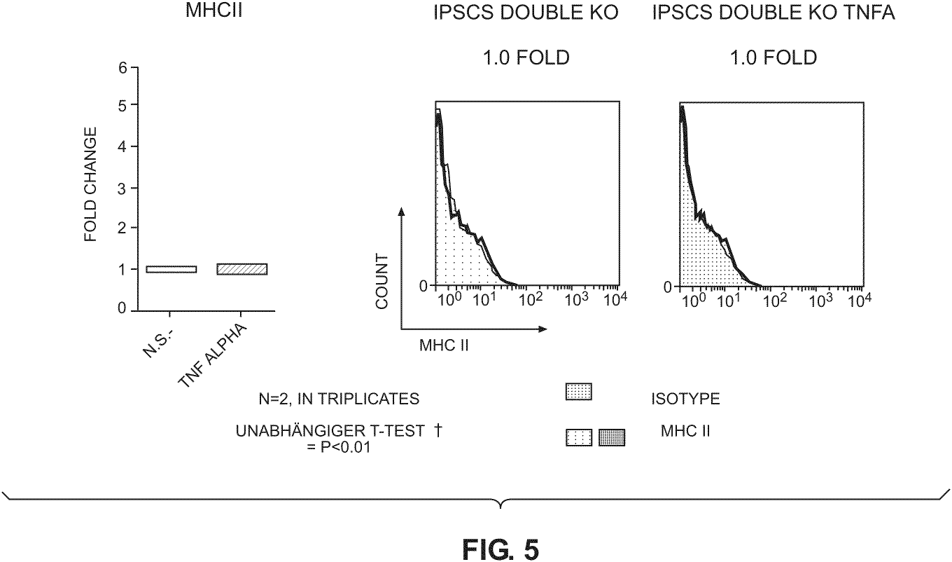

FIG. 5 confirms the miPSC/ß-2-microglobulin/ Ciita double knockout. MHC-II could not be induced by TNF-□ to express MHC-II.

### D. Example 3: Generation of ß-2 Microglobulin/Ciita Double-Knockout-Cd47 Pluripotent Cells

A Cd47 expression vector was introduced into the B2m/Ciita double-knockout miPSC generated above. The vector was delivered using lentivirus containing the antibiotic resistance cassette Blasticidin. The Cd47 gene sequence was synthesized and the DNA was cloned into the plasmid Lentivirus pLenti6/V5 (ThermoFisher, Waltham, MA) containing a blasticidin resistance marker. Sanger sequencing was performed to verify that no mutations has occurred. Lentivirus generation was performed with a stock titer of 1 × 10⁷ TU/ml. The recombinant vector was transduced into 2×10⁵ B2m-KO/Ciita double-knockout mIPSCs, grown on blasticidin resistant MEF cells for 72 h with a MOI ratio of 1:10 followed by antibiotic selection with 12.5 µg/ml Blasticidin for 7 days. Antibiotic selected pools were tested by RT-qPCR amplification of Cd47 mRNA and flow cytometry detection of Cd47. After Cd47 expression was confirmed, the cells were expanded and subjected to pluripotency assays.

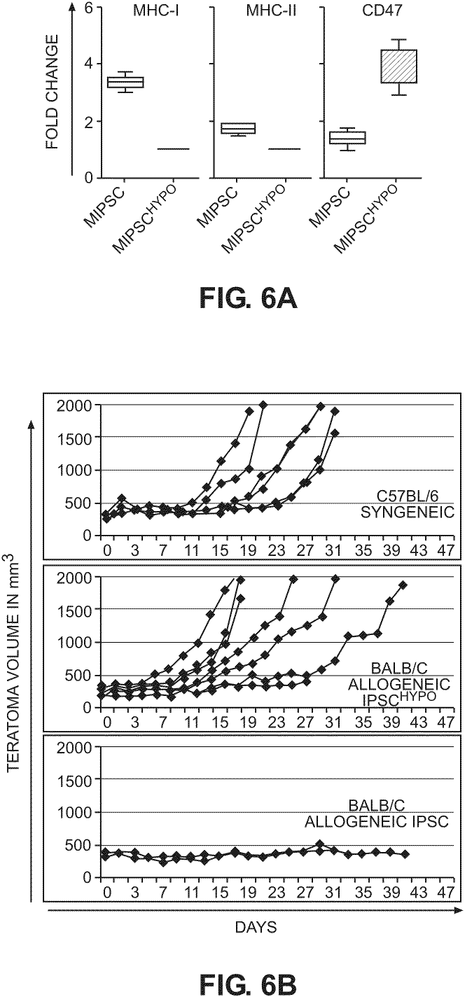

FIG. 6A shows increased Cd47 expression from a transgene added to the ß-2-microglobulin/Ciita double-knockout (iPS^{hypo} cells). FIG. 6B shows that the C57BL/6 iPS^{hypo} cells survive in the allogeneic BALB/c environment but the parental iPS cells do not. This novel result confirms that hypoimmune pluripotent cells survive when transplanted in what would otherwise be incompatible hosts.

### E. Differentiation of Mouse Cells From mHIP Cells

Islet cells: The mHIP cells were differentiated into islet cells using techniques adapted from Liu et al., Exp. Diabetes Res 2012:201295 (doi:10.1155/2012/201295), hereby incorporated by reference and in particular for the differentiation techniques outlined therein. iPS cells were transferred onto gelatin-coated flasks for 30 min to remove the feeder layer and seeded at 1 × 106 cells per well to collagen-I-coated plates in DMEM/F-12 medium supplemented with 2 mM glutamine, 100 µM nonessential amino acids, 10 ng/mL activin A, 10 mM nicotinamide, and 1 µg/mL laminin with 10% FBS overnight. ES-D3 cells were next exposed to DMEM/F-12 medium supplemented with 2 mM L-glutamine, 100 µM nonessential amino acids, 10 ng/mL activin A, 10 mM nicotinamide, 25 µg/mL insulin, and 1 µg/mL laminin with 2% FBS for 6 days.

Neural stem cells: The mHIP cells were differentiated into neural cells using techniques adapted from Abraches et al., doi:10.1371/journal.pone.0006286, hereby incorporated by reference and in particular for the differentiation techniques outlined therein. To start the monolayer protocol, ES cells were plated in serum-free medium ESGRO Complete Clonal Grade medium (Millipore) at high density (1.5×105 cells/cm2). After 24 hours, ES cells were gently dissociated and plated onto 0.1% (v/v) gelatin-coated tissue culture plastic at 1×104 cells/cm2 in RHB-A or N2B27 media (StemCell Science Inc.), changing media every other day. For replating on day 4, cells were dissociated and plated at 2×10⁴ cells/cm2 onto laminin-coated tissue culture plastic in RHB-A medium supplemented with 5 ng/ml murine bFGF (Peprotech). From this point on, cells were replated in the same conditions every 4th day and the medium was changed every 2nd day, for the total of 20 days in culture. To quantify the number of differentiating neurons at each time point, cells were plated onto laminin-coated glass coverslips in 24-well Nunc plates and, 2 days after plating, medium was changed to a RHB-A:Neurobasal:B27 mixture (1:1:0.02), to allow a better survival of differentiated neurons.

Smooth muscle cells: The mHIP cells were differentiated into SM cells using techniques adapted from Huang et al., Biochem Biophys Res Commun 2006:351(2)321-7, hereby incorporated by reference and in particular for the differentiation techniques outlined therein. The resuspended iPSCs were cultivated on 6-well, gelatin coated plastic petri dishes (Falcon, Becton-Dickinson) at 2 mio cell per well at 37 C, 5% CO2 in 2 ml of differentiation medium with the presence of 10 uM atRA, respectively. The differentiation medium was made of DMEM, 15% fetal calf serum, 2 mM L-glutamine, 1 mM MTG (Sigma), 1% nonessential amino acids, penicillin, and streptomycin. The culture was continued for 10 days with daily change of fresh media.

Starting from the 11th day, the differentiation medium was replaced by the serum-free culture medium, which was composed of knock-out DMEM, 15% knock-out serum replacement, 2 mM L-glutamine, 1 mM MTG, 1% nonessential amino acids, penicillin, and streptomycin. The cultures were continued for another 10 days with daily change of the serum-free medium.

Cardiomyocytes: The mHIP cells were differentiated into CM cells using techniques adapted from Kattman et al., Cell Stem Cell 8:228-240 (2011), hereby incorporated by reference and in particular for the differentiation techniques outlined therein.

Endothelial cells: The mHIP cells were differentiated into endothelial cells as known.

### F. Example 4: Allogeneic Transplantation Of HIP Cell Derivatives Show Long-Term Survival In Fully Immunocompetent Recipients

### A. Mice:

BALB/c (BALB/cAnNCrl, H2d), C57BL/6 (C57BL/6J, B6, H2b), BALB/c nude (BALB/c NU/NU, CAnN.CgFoxn1<nu>/Crl, H2d) and Scid beige (CBySmn.CB17-Prkdcscid/J) (all 6-12 weeks) were used as recipients for different assays (all 6-12 weeks of age). The number of animals per experimental group is presented in each figure. Mice were purchased from Charles River Laboratories (Sulzfeld, Germany) and received humane care in compliance with the Guide for the Principles of Laboratory Animals. Animal experiments were approved by the Hamburg “Amt für Gesundheit und Verbraucherschutz” and performed according to local and EU guidelines.

### B. Pluripotency Analysis by RT-PCR and IF:

miPSC were plated in 24-well plates and processed for RT-PCR and immunofluorescence (IF) analysis 48 h after plating. For ICC, cells were fixed, permeabilized and blocked using the Image-iT™ Fixation/Permeabilization Kit (Thermo Fisher Cat. No., R37602). Cells were stained overnight at 4° C. with primary antibodies for Sox2, SSEA-1, Oct4, and Alkaline Phosphatase. After several washes the cells were incubated with an AlexaFluor 488 secondary antibody and NucBlue Fixed Cell ReadyProbes Reagent (all Thermo Fisher Scientific). Stained cells were imaged using a fluorescent microscope.

For RT-PCR, RNA was extracted using the PureLink™ RNA Mini Kit (Thermo Fisher Cat. No. 12183018A). A DNase I step was included to remove contaminating genomic DNA. cDNA was generated using Applied Biosystems® High-Capacity cDNA Reverse Transcription Kit. No-reverse transcriptase (no-RT) controls were also generated from all RNA samples. Gene-specific primers were used to amplify target sequences using AmpliTaq Gol ® 360 Master Mix (Thermo Fisher Cat. No. 4398876). PCR reactions were visualized on 2% agarose gels. A positive control primer set that amplifies a constitutively expressed housekeeping gene (Actb) that encodes a cellular cytoskeleton protein was included.

### C. Gene Editing of Mouse iPSCs:

miPSCs underwent 3 gene-editing steps. First, CRISPRs targeting the coding sequence of mouse B2m gene were annealed and ligated into vectors containing the Cas9 expression cassette. Transfected miPSCs were dissociated to single cells, expanded to colonies, sequenced, and tested for homogenicity. Second, these B2m-/- miPSCs were transfected with vectors containing CRISPRs targeting Ciita, the master regulator of MHC II molecules. Expanded single cell colonies were sequenced and B2m-/- Ciita-/- clones were identified through the presence of aberrant sequence from the CRISPR cleavage site. Third, the Cd47 gene sequence was synthesized and the DNA was cloned into a plasmid lentivirus with a blasticidin resistance. B2m-/- Ciita-/- miPSCs were transfected and grown in the presence of blasticidin. Antibiotic selected pools were tested for Cd47 overexpression and B2m-/- Ciita-/- Cd47 tg miPSCs were expanded. FACS analyses demonstrated high MHC I expression, modest but detectable MHC II expression, and negligible Cd47 expression in wt miPSCs. The lack of MHC I expression, MHC II expression, and Cd47 overexpression in the associated created miPSC lines was confirmed. All engineered miPSC lines were tested for pluripotency. This was confirmed in B2m-/- Ciita-/- Cd47 tg miPSCs after 3 engineering steps and their potential to form cells from all 3 germ layers.

### D. Generation of B2m-/- miPSCs:

CRIPSR technology was used for the knockout of B2m gene. For targeting the coding sequence of mouse beta-2-microglobuline (B2m) gene, the CRISPR sequence 5′-TTCGGCTTCCCATTCTCCGG(TGG)-3′ was annealed and ligated into the All-In-One (AIO) vectors containing the Cas9 expression cassette as per the kit’s instructions (GeneArt CRISPR Nuclease Vector Kit, Thermo Fischer, Waltham, MA). miPSC were transfected with the AIO vectors using Neon electroporation with two 1200 V pulses of 20 ms duration. The transfected iPSC cultures were dissociated to single cells using 0.05% Trypsin (Gibco) and then sorted with FACSAria cell sorter (BD Bioscience, Franklin Lakes, NJ) for removing doublets and debris by selective gating on forward and side scatter emission. Single cells were expanded to full-size colonies and tested for CRISPR edit by screening for presence of aberrant sequence from the CRISPR cleavage site. Briefly, the target sequence was amplified via PCR using AmpliTaq Gold Mastermix (Applied Biosystems, Darmstadt, Germany) and the primers B2m gDNA F: 5′-CTGGATCAGACATATGTGTTGGGA-3′, R: 5′-GCAAAGCAGTTTTAAGTCCACACAG-3′. After cleanup of the obtained PCR product (PureLink® Pro 96 PCR Purification Kit, Thermo Fisher), Sanger sequencing was performed. With the Ion Personal Genome Machine (PGM) Sequencing for the identification of the homogenecity, a 250 bp region of the B2m gene was PCR amplified using primers B2m gDNA PGM F: 5′-TTTTCAAAATGTGGGTAGACTTTGG-3′ and R: 5′-GGATTTCAATGTGAGGCGGGT-3′. The PCR product was purified like previously descript and prepared using the Ion PGM Hi-Q Template Kit (Thermo Fisher). Experiments were performed on the Ion PGM™ System with the Ion 318™ Chip Kit v2 (Thermo Fisher). Analysis for Pluripotency were performed again.

A reduced growth rate or differentiation capacity of B2m-/- iPSCs was not observed as previously reported in the art.

### E. Generation of B2m-/- and Ciita-/- miPSCs:

CRIPSR technology was used for the additional knockout of Ciita gene. For targeting the coding sequence of mouse Ciita gene, the CRISPR sequence 5′-GGTCCATCTGGTCATAGAGG (CGG)-3′ was annealed and ligated into the All-In-One (AIO) vectors containing the Cas9 expression cassette as per the kit’s instructions (GeneArt CRISPR Nuclease Vector Kit, Thermo Fischer, Waltham, MA). miPSC were transfected with the AIO vectors using the same condition for B2m-KO. The transfected miPSC cultures were dissociated to single cells using 0.05% Trypsin (Gibco) and then sorted with FACSAria cell sorter (BD Bioscience, Franklin Lakes, NJ) for removing doublets and debris by selective gating on forward and side scatter emission. Single cells were expanded to full-size colonies and tested for CRISPR edit by screening for presence of aberrant sequence from the CRISPR cleavage site. Briefly, the target sequence was amplified via PCR using AmpliTaq Gold Mastermix (Applied Biosystems, Darmstadt, Germany) and the primers Ciita gDNA F: 5′-CCCCCAGAACGATGAGCTT-3′, R: 5′-TGCAGAAGTCCTGAGAAGGCC-3′. After cleanup of the obtained PCR product (PureLink® Pro 96 PCR Purification Kit, Thermo Fisher), Sanger sequencing was performed. Using the DNA sequence chromatogram, edited clones were then identified through the presence of aberrant sequence from the CRISPR cleavage site. Indel size was calculated using the TIDE tool. PCR and ICC were performed again to verify the pluripotency status of the cells.

### F. Generation of B2m-/- Ciita-/- Cd47 Tg miPSCs:

The cell line B2m-KO, Ciita-KO and Cd47-tg iPSC was generated through antibiotic resistance selection after lenitvirus mediated delivery of a Cd47 expression vector containing the antibiotic resistance cassette Blasticidin. The Cd47 gene sequence was synthesized and the DNA was cloned into the plasmid Lentivirus pLenti6/V5 (ThermoFisher) with a blasticidin resistance. Sanger sequencing was performed to verify that no mutation has occurred. Lentivirus generation was performed with a stock titer of 1 × 10⁷ TU/ml. The transduction was perfomed into 2×10⁵ B2m-/- Ciita-/- miPSCs, grown on blasticidin resistant MEF cells for 72 h with a MOI ratio of 1:10 followed by antibiotic selection with 12.5 µg/ml Blasticidin for 7 days. Antibiotic selected pools were tested by RT-qPCR amplification of Cd47 mRNA and flow cytometry detection of Cd47. After the confirmation of Cd47, cells were expanded and confirmed by running pluripotency assays.

### G. Derivation and Characterization of iPSC-Derived Endothelial Cells (iECs):

iECs were derived using a three-dimensional approach. Briefly, to initiate differentiation, iPSCs were cultured in ultra-low, non-adhesive dishes to form embryoid body (EB) aggregates in EBM2 media (Lonza) in the absence of leukemia inhibitor factor (LIF). After 4 days of suspension culture, the EBs were reattached onto 0.2% gelatin-coated dishes and cultured in EBM2 medium supplemented with VEGF-A165 (50 ng/mL; PeproTech). After 3 weeks of differentiation, single cell suspensions were obtained using a cell dissociation buffer (Life Technologies) and labeled with APC-conjugated CD31 (eBiosciences) and PE-conjugated CD144 (BD Biosciences) anti-mouse antibodies. iECs were purified by fluorescence activated cell sorting (FACS) of CD31+CD144+ population. iECs were maintained in EBM2 media supplemented with recombinant murine vascular endothelial growth factor (50 ng/ml).

Their phenotype was confirmed by immunofluorescense for CD31 and VE cadherin, as well as by PCR and tube formation assays to demonstrate endothelial function to form premature vascular structures. Note: Differentiation protocols using confluent iPSC monolayers on 0.1% gelatine or Matrigel have also been successful. Note: Other endothelial cell media have been also successfully used.

### H. Derivation and Characterization of iPSC-Derived Smooth Muscle Cells (iSMCs):

The resuspended iPSCs were cultivated on 6-well, 0.1% gelatin coated plastic petri dishes (Falcon, Becton-Dickinson) at 2 mio cell per well at 37 C, 5% CO₂ in 2 ml of differentiation medium with the presence of 10 uM. The differentiation medium was made of DMEM, 15% fetal calf serum, 2 mM L-glutamine, 1 mM MTG (Sigma), 1% nonessential amino acids, penicillin, and streptomycin. The culture was continued for 10 days with daily media changes.

Starting from the 11th day, the differentiation medium was replaced with a serum-free culture medium of a knock-out DMEM: 15% knock-out serum replacement, 2 mM L-glutamine, 1 mM MTG, 1% nonessential amino acids, penicillin, and streptomycin. The cultures were continued for another 10 days with daily changes of the serum-free medium. The phenotype was confirmed by immunofluorescence and PCR for both, SMA and SM22.

### I. Derivation and Characterization of iPSC-Derived Cardiomyocytes (iCMs):

Prior to differentiation, iPSCs were passaged two times on gelatin-coated dishes to remove the feeder cells. In brief, iPSCs were dissociated with TrypLE (Invitrogen) and cultured at 75,000-100,000 cells/ml without any additional growth factors for 48 hr. The 3-day-old EBs were dissociated and the cells were differentiated in “cardiac conditions”. In brief, 6 × 10⁴-10 × 10⁴ cells were seeded into individual wells of a 96-well flat bottom plate (Becton Dickenson, Franklin Lakes, NJ) coated with gelatin in StemPro-34 SF medium (Invitrogen), supplemented with 2 mM L-glutamine, 1 mM ascorbic acid (Sigma), human-VEGF (5 ng/ml), human-DKK1 (150 ng/ml), human bFGF (10 ng/ml), and human FGF10 (12.5 ng/ml) (R&D Systems). Cultures were harvested 4 or 5 days later (total of 7 or 8 days).

Their phenotype was confirmed by IF for troponin I and sarcomeric alpha actinin as well as PCR for Gata4 and Mhy6. The cells started beating between 8-10 days. This demonstrated their functional differentiation.

### J. Derivation and Characterization of iPSC-Derived Islet Cells (iICs)

iPS cells were transferred onto gelatin-coated flasks for 30 min to remove the feeder layer and seeded at 1 × 10⁶ cells per well to collagen-I-coated plates in DMEM/F-12 medium supplemented with 2 mM glutamine, 100 µM nonessential amino acids, 10 ng/mL activin A, 10 mM nicotinamide, and 1 µg/mL laminin with 10% FBS overnight. ES-D3 cells were next exposed to DMEM/F-12 medium supplemented with 2 mM L-glutamine, 100 µM nonessential amino acids, 10 ng/mL activin A, 10 mM nicotinamide, 25 µg/mL insulin, and 1 µg/mL laminin with 2% FBS for 6 days. Their phenotype was confirmed by immunofluorescence for c-peptide, PCR for glucagon, Ngn3, amylase, insulin 2, somatostatin and insulin production.

### K. Derivation and Characterization of iPSC-Derived Neuronal Cells (iNCs)

To start the monolayer protocol, iPSCs were gently dissociated and plated onto 0.1% gelatin-coated tissue culture plastic at 1×10⁴ cells/cm² in RHB-A or N2B27 media (StemCell Science Inc.), changing media every other day. For replating on day 4, cells were dissociated and plated at 2×10⁴ cells/cm² onto laminin-coated tissue culture plastic in RHB-A medium supplemented with 5 ng/ml murine bFGF (Peprotech). From this point on, cells were replated in the same conditions every 4th day and the medium was changed every 2nd day, for the total of 20 days in culture. To quantify the number of differentiating neurons at each time point, cells were plated onto laminin-coated glass coverslips in 24-well Nunc plates and, 2 days after plating, medium was changed to a RHB-A:Neurobasal:B27 mixture (1:1:0.02), to allow a better survival of differentiated neurons. Their phenotype was confirmed by IF for Tuj-1 and nestin.

### L. Elispot Assays

For uni-directional Enzyme-Linked ImmunoSpot (Elispot) assays, recipient splenocytes were isolated from fresh spleen 5 days after cell injection (miPSC, miPSC B2m-/- or miPSC B2m-/- Ciita-/- or miPSC B2m-/- Ciita-/- Cd47 tg) and used as responder cells. Donor cells (miPSC, miPSC B2m-/- or miPSC B2m-/- Ciita-/- or miPSC B2m-/- Ciita-/-Cd47 tg) were mitomycin-inhibited and served as stimulator cells. 10⁶ stimulator cells were incubated with 5×10⁵ recipient responder splenocytes for 24 h and IFNγ and IL-4 spot frequencies were automatically enumerated using an Elispot plate reader. Quadruplicates were performed in all assays.

### M. Teratoma Assays to Study iPSC Survival in Vivo

Six-week old syngeneic or allogeneic mice were used for transplantation of wtiPSCs or non-immunogenic iPSCs. 1×10⁶ cells were injected in 100 µl into the right thigh muscle of the mice. The transplanted animals were observed routinely every other day, and tumor growth was measured with a caliper. They were sacrificed after development of tumors larger than 1.5 cm³ or following an observation period of 100 days.

### N. NK Cell Assays in Vitro

CD107 expression on NK cells after co-culture with wt iPSCs or HIP cells was measured by flow cytometry as NK cell activation marker. Using the Elispot principle, NK cells were co-cultured with wt iPSCs or HIP cells and their IFN-γ release was measured.

According to the ‘missing self theory’, MHC I-deficient stem cells have been demonstrated to be susceptible to NK killing as both murine and human PSCs express ligands for activating NK receptors. Although the expression of activating receptors has been reported to decrease with differentiation, NK killing of B2m-/- derivatives has been observed. Although isolated expression of HLA-E or HLA-G in human pluripotent stem cells has been used to mitigate the expected innate immune response in HLA I-/- cells, there are very effective additional inhibitory non-MHC ligands among them. The invention provides that Cd47 was found to be a surprisingly potent inhibitor of innate immune clearance.

### O. Summary of the Mouse Data

All engineered miPSC lines were transplanted into syngeneic C57BL/6 and allogeneic BALB/c recipients without any immunosuppression. While all engineered cells similarly developed teratomas in syngeneic recipients, their survival depended on their level of hypo-immunogenicity in allogeneic recipients. 60% teratoma formation of B2m-/- miPSCs in BALB/c, a subtle Elispot response, and still a measurable IgM antibody response was observed. In B2m-/- Ciita-/- miPSCs 91.7% teratoma formation in allogeneic BALB/c, a minor Elispot response, and no antibody response was seen. The final B2m-/- Ciita-/- Cd47 tg miPSC line showed 100% teratoma formation, and no Elispot or antibody responses. The contribution of the Cd47 overexpression was additionally evaluated in assays of innate immunity by comparing B2m-/- Ciita-/- miPSCs with B2m-/- Ciita-/- Cd47 tg miPSCs. Cd47 overexpression significantly reduced NK cell CD107 expression and NK cell IFN-γ release, thus mitigating innate immune clearance. In summary, every engineering step has made the miPSCs more hypo-immunogenic.

B2m-/- Ciita-/- HIP cells differentiated into hypo-immunogenic endothelial-like cells (miECs), smooth muscle-like cells (miSMCs), and cardiomyocyte-like cells (miCMs). “Wild type” miPSC-derivatives (i.e. from non-engineered miPSCs) served as controls. All derivatives showed the typical morphologic appearance, cell marker immunofluorescence and gene expression of their intended mature tissue cell lines. The expression of MHC I and II molecules in wt derivatives was generally largely upregulated compared to their parental miPSC line, but markedly varied by cell type. As expected, miECs had by far the highest expression of MHC I and MHC II, miSMCs had moderate MHC I and MHC II expression, while miCMs had moderate MHC I but very low MHC II expression. All wt derivatives had rather low Cd47 expression, although also mildly up compared to miPSCs. All B2m-/- Ciita-/- Cd47 tg derivatives appropriately showed a complete lack of MHC I and MHC II and significantly higher Cd47 than their wt counterparts.

Matrigel plugs containing 5×10⁵ wt miECs, miSMCs, and miCMs were transplanted into subcutaneous pouches of syngeneic C57BL/6 or allogeneic BALB/c mice. After 5 days, all allogeneic recipients mounted a strong cellular immune response as well as strong IgM antibody response against these differentiated wt cell grafts. In sharp contrast, neither of the corresponding B2m-/- Ciita-/- Cd47 tg (HIP) derivatives showed detectable increases in IFN-γ Elispot frequencies or IgM antibody production.

The morphology of the transplanted cells was also confirmed. Allogeneic mice were placed in an induction chamber and anaesthesia was induced with 2% isoflurane (Isothesia, Butler Schein). 1 mio cells, either HIP miPSC-derived cardiomyocytes (miCM), HIP miPSC-derived smooth muscle cells (miSMC) or HIP miPSC-derived endothelial cells (miEC) in 250 ul 0.9% saline were mixed with 250 ul BD Matrigel High Concentration (1:1; BD Biosciences) and injected subcutaneously in the lower dorsa of mice using a 23-G syringe. Matrigel plugs were explantated 1, 2, 3, 4, 5, 6, 8, 10 and 12 weeks after implantation and were fixed with 4% paraformaldehyde and 1% Glutenaldehyde for 24 h, followed by dehydration and embedding in paraffin. Sections of 5 µm thickness were cut and stained with Hematoxylin and Eosin (HE). Histology confirmed morphologically-adequate miCMs, miSMCs, and miECs.

### G. Example 5: Generation of Human iPSCs

The Human Episomal iPSC Line was derived from CD34+ cord blood (Cat. No. A33124, Thermo Fisher Scientific) using a three-plasmid, seven-factor (SOKMNLT; SOX2, OCT4 (POU5F1), KLF4, MYC, NANOG, LIN28, and SV40L T antigen) EBNA-based episomal system from ThermoFisher. This iPSC line is considered to have a zero footprint as there was no integration into the genome from the reprogramming event. It has been shown to be free of all reprogramming genes. The iPSCs have a normal XX karyotype and endogenous expression of pluripotent markers like Oct4, Sox2, Nanog (as shown by RT-PCR) Oct4, SSEA4, TRA-1-60 and TRA-1-81 (as shown by ICC). In directed differentiation and teratoma analyses, these hiPSCs retained their differentiation potential for the ectodermal, endodermal, and mesodermal lineages. In addition, vascular, endothelial, and cardiac lineages were derived with robust efficiencies.

Note: Several gene-delivery vehicles for iPSC generation were successfully used, including retroviral vectors, adenoviral vectors, Sendai virus as well as virus-free reprogramming methods (using episomal vectors, piggyBac transposon, synthetic mRNAs, microRNAs, recombinant proteins, and small molecule drugs, etc).

Note: Different factors were successfully used for re-programming, such as the first reported combination of OCT3/4, SOX2, KLF4, and C-MYC, known as the Yamanaka factors. In one embodiment, only three of these factors were successfully combined and omittied C-MYC, although with reduced reprogramming efficiency.

In one embodiment, L-MYC or GLIS1 instead of C-MYC showed improved reprogramming efficiency. In another embodiment, reprogramming factors are not limited to genes associated with pluripotency.

### A. Statistics

All data are expressed as mean ± SD or in box blot graphs showing the median and the minimum to maximum range. Intergroup differences were appropriately assessed by either the unpaired Student’s t test or the one-way analysis of variance (ANOVA) with Bonferroni’s postHoc test. * p<0.05, ** p<0.01.

### H. Example 6: Generation of Human HIP Cells

Human Cas9 iPSC underwent 2 gene-editing steps. In the first step, CRISPR technology was performed by a combined targeting of the coding sequence of human beta-2-microglobuline (B2M) gene with the CRISPR sequence 5′-CGTGAGTAAACCTGAATCTT-3′ and the coding sequence of human CIITA gene with the CRISPR sequence 5′-GATATTGGCATAAGCCTCCC-3′. Linearized CRISPR sequence with T7 promoter was used to synthesize gRNA as per the kit’s instructions (MEGAshortscript T7 Transcription Kit, Thermo Fisher). The collected in-vitro transcription (IVT) gRNA was then purified via the MEGAclear Transcription Clean-Up Kit. For IVT gRNA delivery, singularized cells were electroporated with 300 ng IVT gRNA using a Neon electroporation system. After electroporation, edited Cas9 iPSCs were expanded for single cell seeding: iPSC cultures were dissociated to single cells using TrypLE (Gibco) and stained with Tra1-60 Alexa Fluor® 488 and propidium iodide (PI). FACS Aria cell sorter (BD Biosciences) was used for the sorting and doublets and debris were excluded from seeding by selective gating on forward and side scatter emission. Viable pluripotent cells were selected on the absence of PI and presence of Tra1-60 Alexa Fluor 488 staining. Single cells were then expanded into full size colonies, after which the colonies were tested for a CRISPR edit. CRISPR mediated cleavage was assessed using the GeneArt Genomic Cleavage Detection Kit (Thermo Fisher). Genomic DNA was isolated from 1×10⁶ hiPSCs and the B2M and CIITA genomic DNA regions were PCR amplified using AmpliTaq Gold 360 Master Mix and the primer sets F: 5′-TGGGGCCAAATCATGTAGACTC -3′ and R: 5′-TCAGTGGGGGTGAATTCAGTGT-3′ for B2M as well as F: 5′-CTTAACAGCGATGCTGACCCC-3′ and R: 5′-TGGCCTCCATCTCCCCTCTCTT-3′ for CIITA. For TIDE analysis, the obtained PCR product was cleaned up (PureLink PCR Purification Kit, Thermo Fisher) and Sanger sequencing was performed for the prediction of indel frequency. After the confirmation of B2M/CIITA knockout, cells were further characterized through karyotype analysis and the TaqMan hPSC Scorecard Panel (Thermo Fisher). The PSC were found to be pluripotent and maintained a normal (46, XX) karyotype during the genome editing process.

In the second step, the CD47 gene was synthesized and the DNA was cloned into a plasmid lentivirus with an EF1a promotor and puromycin resistance. Cells were transduced with lentiviral stocks of 1×10⁷ TU/mL and 6 µg/mL of Polybrene (Thermo Fisher). Media was changed daily after transduction. Three days after transduction, cells were expanded and selected with 0.5 µg/mL of puromycin. After 5 days of antibiotic selection, antibiotic resistant colonies emerged and were further expanded to generate stable pools. Level of CD47 was confirmed by qPCR. Pluripotency assay (TaqMan hPSC Scorecard Panel, Thermo Fisher). and karyotyping were performed again to verify the pluripotent status of the cells.

### I. Example 7: Differentiation of Human HIP Cells

### 1. Differentiation of hHIP Cells to Human Cardiomyocytes

This was done using a protocol adapted from Sharma et al., J. Vis Exp. 2015 doi: 10.3791/52628, hereby incorporated by reference in its entirety and specifically for the techniques to differentiate the cells. hiPSCs were plated on diluted Matrigel (356231, Corning) in 6-well plates and maintained in Essential 8 Flex media (Thermo Fisher). After the cells arrived at 90% confluency, the differentiation was started and media was changed to 5 mL of RPMI1640 containing 2% B-27 minus Insulin (both Gibco) and 6uM CHIR-99021 (Selleck Chem). After 2 days, media was changed to RPMI1640 containing 2% B-27 minus Insulin without CHIR. On day 3, 5uL IWR1 was added to the media for two further days. At day 5, the media was changed back to RPMI 1640 containing 2% B-27 minus insulin medium and incubated for 48 hr. At day 7, media was changed to RPMI 1640 containing B27 plus insulin (Gibco) and replaced every 3 days thereafter with the same media. Spontaneous beating of cardiomyocytes was first visible at approximately day 10 to day 12. Purification of Cardiomyocytes was performed on day 10 post-differentiation. Briefly, media was changed to low glucose media and maintained for 3 days. At day 13, media was changed back to RPMI 1640 containing B27 plus insulin. This procedure was repeated on day 14. The remaining cells are highly purified cardiomyocytes.

### 2. Differentiation of hHIP Cells to Human Endothelial Cells

hiPSC were plated on diluted Matrigel (356231, Corning) in 6-well plates and maintained in Essential 8 Flex media (Thermo Fisher). After the cells arrived at 60% confluency, the differentiation was started and media was changed to RPMI1640 containing 2% B-27 minus Insulin (both Gibco) and 5 µM CHIR-99021 (Selleck Chem). On day 2, the media was changed to reduced media: RPMI1640 containing 2% B-27 minus Insulin (both Gibco) and 2 µM CHIR-99021 (Selleck Chem). From day 4 to day 7, cells were exposed to RPMI EC media, RPMI1640 containing 2% B-27 minus Insulin plus 50 ng/mL vascular endothelial growth factor (VEGF; R&D Systems, Minneapolis, MN, USA), 10 ng/mL fibroblast growth factor basic (FGFb; R&D Systems), 10 µM Y-27632 (Sigma-Aldrich, Saint Louis, MO, USA) and 1 µM SB 431542 (Sigma-Aldrich). Endothelial cell clusters were visible from day 7 and cells were maintained in EGM-2 SingleQuots media (Lonza, Basel, Switzerland) plus 10% FCS hi (Gibco), 25 ng/mL vascular endothelial growth factor (VEGF; R&D Systems, Minneapolis, MN, USA), 2 ng/mL fibroblast growth factor basic (FGFb; R&D Systems), 10 µM Y-27632 (Sigma-Aldrich, Saint Louis, MO, USA) and 1 µM SB 431542 (Sigma-Aldrich). The differentiation process completed after 14 days und undifferentiated cells detached during the differentiation process. For purification, cells went through MACS progress according to the manufactures’ protocol using CD31 microbeads (Miltenyi, Auburn, CA). The highly purificated EC-cells were cultured in EGM-2 SingleQuots media (Lonza, Basel, Switzerland) plus supplements and 10% FCS hi (Gibco). TrypLE was used for passaging the cells 1:3 every 3 to 4 days.

### J. Transplantation in Humanized Mice

Humanized NSG-SGM3 mice were placed in an induction chamber and anaesthesia was induced with 2% isoflurane (Isothesia, Butler Schein). 1 mio cells, either hiPSC-derived cardiomyocytes (hiCM) or hiPSC-derived endothelial cells (hiECin 250 ul 0.9% saline containing ZVAD (100 mM, benzyloxycarbonyl-Val-Ala-Asp(O-methyl)-fluoromethyl ketone, Calbiochem), Bcl-XL BH4 (cell-permeant TAT peptide, 50 nM, Calbiochem), cyclosporine A (200 nM, Sigma), IGF-1 (100 ng/ml, Peprotech) and pinacidil (50 mM, Sigma) were mixed with 250 ul BD Matrigel High Concentration (1:1; BD Biosciences) and injected subcutaneously in the lower dorsa of mice using a 23-G syringe. Matrigel plugs were explanted 2, 4, 6, 8, 10 and 12 weeks after implantation and were fixed with 4% paraformaldehyde and 1% Glutenaldehyde for 24 h, followed by dehydration and embedding in paraffin. Section of 5 µm thickness were cut and stained with Hematoxylin and Eosin (HE) and the morphology was confirmed.

### IX. EXEMPLARY SEQUENCES

SEQ ID NO:1 - Human β-2-Microglobulin

MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDI

SEQ ID NO:2 - Human CIITA protein, 160 amino acid N-terminus

MRCLAPRPAGSYLSEPQGSSQCATMELGPLEGGYLELLNSDADPLCLYHF
YDQMDLAGEEEIELYSEPDTDTINCDQFSRLLCDMEGDEETREAYANIAE
LDQYVFQDSQLEGLSKDIFKHIGPDEVIGESMEMPAEVGQKSQKRPFPEE
LPADLKHWKP

SEQ ID NO:3 - Human CD47

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVE

SEQ ID NO:4 - Herpes Simplex Virus Thimidine Kinase (HSV-tk)

MASYPCHQHASAFDQAARSRGHSNRRTALRPRRQQEATEVRLEQKMPTLL
RVYIDGPHGMGKTTTTQLLVALGSRDDIVYVPEPMTYWQVLGASETIANI
YTTQHRLDQGEISAGDAAVVMTSAQITMGMPYAVTDAVLAPHVGGEAGSS
HAPPPALTLIFDRHPIAALLCYPAARYLMGSMTPQAVLAFVALIPPTLPG
TNIVLGALPEDRHIDRLAKRQRPGERLDLAMLAAIRRVYGLLANTVRYLQ
GGGSWWEDWGQLSGTAVPPQGAEPQSNAGPRPHIGDTLFTLFRAPELLAP
NGDLYNVFAWALDVLAKRLRPMHVFILDYDQSPAGCRDALLQLTSGMVQT
HVTTPGSIPTICDLARTFAREMGEAN

SEQ ID NO:5 - Escherichia coli Cytosine Deaminase (EC-CD)

MSNNALQTIINARLPGEEGLWQIHLQDGKISAIDAQSGVMPITENSLDAE
QGLVIPPFVEPHIHLDTTQTAGQPNWNQSGTLFEGIERWAERKALLTHDD
VKQRAWQTLKWQIANGIQHVRTHVDVSDATLTALKAMLEVKQEVAPWIDL
QIVAFPQEGILSYPNGEALLEEALRLGADVVGAIPHFEFTREYGVESLHK
TFALAQKYDRLIDVHCDEIDDEQSRFVETVAALAHHEGMGARVTASHTTA
MHSYNGAYTSRLFRLLKMSGINFVANPLVNIHLQGRFDTYPKRRGITRVK
EMLESGINVCFGHDDVFDPWYPLGTANMLQVLHMGLHVCQLMGYGQINDG
LNLITHHSARTLNLQDYGIAAGNSANLIILPAENGFDALRRQVPVRYSVR
GGKVIASTQPAQTTVYLEQPEAIDYKR

SEQ ID NO:6 - Truncated human Caspase 9

GFGDVGALESLRGNADLAYILSMEPCGHCLIINNVNFCRESGLRTRTGSN
IDCEKLRRRFSSLHFMVEVKGDLTAKKMVLALLELAQQDHGALDCCVVVI
LSHGCQASHLQFPGAVYGTDGCPVSVEKIVNIFNGTSCPSLGGKPKLFFI
QACGGEQKDHGFEVASTSPEDESPGSNPEPDATPFQEGLRTFDQLDAISS
LPTPSDIFVSYSTFPGFVSWRDPKSGSWYVETLDDIFEQWAHSEDLQSLL
LRVANAVSVKGIYKQMPGCFNFLRKKLFFKTS

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1-9. (canceled)

10. A hypoimmunogenic cell, comprising:
a. one or more genetic alterations that reduce expression of a functional Major Histocompatibility Antigen Class I (HLA-I) complex as compared to a parental cell, and/or
one or more genetic alterations that reduce expression of a functional Major Histocompatibility Antigen Class II (HLA-II) complex as compared to a parental cell; and
b. one or more genetic alterations that increase expression of a functional CD47 protein as compared to the parental cell,
wherein the hypoimmunogenic cell is suitable for therapeutic administration to a subject once every 2 weeks or longer.

11-88. (canceled)

89. The hypoimmunogenic cell of claim 10, wherein the hypoimmunogenic cell is suitable for therapeutic administration to a subject once every 4 weeks or longer, or once every 6 weeks or longer.

90. The hypoimmunogenic cell of claim 10, wherein the hypoimmunogenic cell is an islet cell, a neuronal cell, a neuronal stem cell, a smooth muscle cell, a cardiomyocyte, an endothelial cell, an endothelial colony forming cell, a thyroid cell, a thyroid progenitor cell, or a retinal pigment epithelium (RPE) cell.

91. The hypoimmunogenic cell of claim 10, further comprising a safety switch comprising a suicide gene that is:
(i) a herpes simplex virus thymidine kinase gene (HSV-tk);
(ii) an Escherichia coli cytosine deaminase gene (EC-CD); or
(iii) an inducible Caspase protein.

92. The hypoimmunogenic cell of claim 10, wherein the hypoimmunogenic cell is less susceptible than the parent cell to rejection when transplanted into a subject, wherein rejection comprises macrophage phagocytosis, Natural Killer (NK) cell response, or both.

93. The hypoimmunogenic cell of claim 10, wherein the hypoimmunogenic cell is derived from a pluripotent cell, an induced pluripotent stem cell (iPSC), an embryonic stem cell, a fetal stem cell, an amniotic stem cell, or a somatic stem cell.

94. The hypoimmunogenic cell of claim 10, wherein the one or more genetic alterations that reduce expression of a functional HLA-I complex comprise one or more genetic alterations that reduce or knock-out expression of one or more HLA-I genes or HLA-I associated genes as compared to a parental cell.

95. The hypoimmunogenic cell of claim 94, wherein the one or more genetic alterations that reduce expression of a functional HLA-I complex are present on both alleles of the one or more HLA-I genes or HLA-I associated genes.

96. The hypoimmunogenic cell of claim 94, wherein the one or more HLA-I genes comprise an HLA-A gene, an HLA-B gene, an HLA-C gene, or a combination thereof.

97. The hypoimmunogenic cell of claim 94, wherein the one or more HLA-I associated genes comprise a β-2 microglobulin (B2M) gene.

98. The hypoimmunogenic cell of claim 10, wherein the one or more genetic alterations that reduce expression of a functional HLA-II complex comprise one or more genetic alterations that reduce or knock-out expression of one or more HLA-II genes or HLA-II associated genes as compared to a parental cell.

99. The hypoimmunogenic cell of claim 98, wherein the one or more genetic alterations that reduce expression of a functional HLA-II complex are present on both alleles of the one or more HLA-II genes or HLA-II associated genes.

100. The hypoimmunogenic cell of claim 98, wherein the one or more HLA-II genes comprise an HLA-DP gene, an HLA-DR gene, an HLA-DQ gene, or a combination thereof.

101. The hypoimmunogenic cell of claim 98, wherein the one or more HLA-II associated genes comprise a CIITA gene.

102. The hypoimmunogenic cell of claim 10, wherein expression of one or more HLA-I genes or HLA-I associated genes comprises surface expression of one or more proteins encoded by the one or more HLA-I genes.

103. The hypoimmunogenic cell of claim 10, wherein expression of one or more HLA-II genes or HLA-II associated genes comprises surface expression of one or more proteins encoded by the one or more HLA-II genes.

104. The hypoimmunogenic cell of claim 10, wherein the one or more genetic alterations that increase expression of CD47 protein comprises expression of a CD47 transgene or a modification to an endogenous CD47 gene locus.

105. The hypoimmunogenic cell of claim 104, wherein the one or more genetic alterations that increase expression of CD47 protein comprises:
(i) an exchange of an endogenous promoter for a constitutive or inducible promoter; or
(ii) a CD47 transgene under the control of an inducible or constitutive promoter.

106. The hypoimmunogenic cell of claim 10, wherein one or more of the genetic alterations are introduced to the cell using a zinc finger nuclease (ZFN), transcription activator-like effector nuclease (TALEN), clustered regularly interspaced short palindromic repeats (CRISPR)/nuclease, a retroviral vector, a lentiviral vector, an adenovirus vector, a Sendai viral vector, or a combination thereof.

107. The hypoimmunogenic cell of claim 10, wherein the hypoimmunogenic cell is human.

108. The hypoimmunogenic cell of claim 10, wherein the level of CD47 protein expressed in the hypoimmunogenic cell is 1.5-fold to 5-fold greater than the level of CD47 protein expressed in the parental cell.

109. A hypoimmunogenic cell, comprising:
a. one or more genetic alterations that reduce expression of a functional Major Histocompatibility Antigen Class I (HLA-I) complex as compared to a parental cell, and/or one or more genetic alterations that reduce expression of a functional Major Histocompatibility Antigen Class II (HLA-II) complex as compared to a parental cell; and
b. one or more genetic alterations that increase expression of a functional CD47 protein as compared to the parental cell,
wherein the level of CD47 protein expressed in the hypoimmunogenic cell is 1.5-fold to 5-fold greater than the level of CD47 protein expressed in the parental cell.

110. A method of producing a hypoimmunogenic cell of claim 10, comprising:
a. introducing into the parent cell one or more genetic alterations that reduce expression of a functional Major Histocompatibility Antigen Class I (HLA-I) complex as compared to a parental cell, and/or one or more genetic alterations that reduce expression of a functional Major Histocompatibility Antigen Class II (HLA-II) complex as compared to a parental cell; and
b. introducing into the parent cell one or more genetic alterations that increase expression of a functional CD47 protein as compared to a parental cell.

111. A method of treating a disease or disorder in a subject, wherein the method comprises administering a hypoimmunogenic cell of claim 10 to the subject.

112. The method of claim 111, wherein the disease or disorder is a disease or disorder of the eye, liver, heart, lung, kidney, pancreas, brain, neural tissue, blood, bone, thyroid, vasculature or bone marrow.

113. The method of claim 111, wherein the disease or disorder is an eye disease, a cardiovascular disease, a peripheral arterial disease, diabetes, a loss of hepatocyte functioning, liver cirrhosis, or autoimmune thyroiditis.

114. The method of claim 111, wherein administering comprises transplanting the hypoimmunogenic cell once every about 2 weeks or longer.


SEQUENCE LISTING (22 sequences)

SEQ ID NO: 1 - protein, 119 aa, Homo sapiens [sequence omitted]

SEQ ID NO: 2 - protein, 160 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKP

SEQ ID NO: 3 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 4 - protein, 376 aa, unidentified
  source 1..376: note = Herpes simplex virus
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 6 - protein, 282 aa, Homo sapiens
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 7 - DNA, 23 bp, synthetic construct
  misc_feature 1..23: note = Description of Artificial Sequence: Synthetic oligonucleotide
  source 1..23: mol_type = other DNA
ttcggcttcccattctccggtgg

SEQ ID NO: 8 - DNA, 22 bp, synthetic construct
  misc_feature 1..22: note = Description of Artificial Sequence: Synthetic oligonucleotide
  source 1..22: mol_type = other DNA
gtatactcacgccacccaccgg

SEQ ID NO: 9 - DNA, 23 bp, synthetic construct
  misc_feature 1..23: note = Description of Artificial Sequence: Synthetic oligonucleotide
  source 1..23: mol_type = other DNA
ggcgtatgtatcagtctcagtgg

SEQ ID NO: 10 - DNA, 24 bp, synthetic construct
  misc_feature 1..24: note = Description of Artificial Sequence: Synthetic primer
  source 1..24: mol_type = other DNA
ctggatcagacatatgtgttggga

SEQ ID NO: 11 - DNA, 25 bp, synthetic construct
  misc_feature 1..25: note = Description of Artificial Sequence: Synthetic primer
  source 1..25: mol_type = other DNA
gcaaagcagttttaagtccacacag

SEQ ID NO: 12 - DNA, 25 bp, synthetic construct
  misc_feature 1..25: note = Description of Artificial Sequence: Synthetic primer
  source 1..25: mol_type = other DNA
ttttcaaaatgtgggtagactttgg

SEQ ID NO: 13 - DNA, 21 bp, synthetic construct
  misc_feature 1..21: note = Description of Artificial Sequence: Synthetic primer
  source 1..21: mol_type = other DNA
ggatttcaatgtgaggcgggt

SEQ ID NO: 14 - DNA, 23 bp, synthetic construct
  misc_feature 1..23: note = Description of Artificial Sequence: Synthetic oligonucleotide
  source 1..23: mol_type = other DNA
ggtccatctggtcatagaggcgg

SEQ ID NO: 15 - DNA, 19 bp, synthetic construct
  misc_feature 1..19: note = Description of Artificial Sequence: Synthetic primer
  source 1..19: mol_type = other DNA
cccccagaacgatgagctt

SEQ ID NO: 16 - DNA, 21 bp, synthetic construct
  misc_feature 1..21: note = Description of Artificial Sequence: Synthetic primer
  source 1..21: mol_type = other DNA
tgcagaagtcctgagaaggcc

SEQ ID NO: 17 - DNA, 20 bp, synthetic construct
  misc_feature 1..20: note = Description of Artificial Sequence: Synthetic oligonucleotide
  source 1..20: mol_type = other DNA
cgtgagtaaacctgaatctt

SEQ ID NO: 18 - DNA, 20 bp, synthetic construct
  misc_feature 1..20: note = Description of Artificial Sequence: Synthetic oligonucleotide
  source 1..20: mol_type = other DNA
gatattggcataagcctccc

SEQ ID NO: 19 - DNA, 22 bp, synthetic construct
  misc_feature 1..22: note = Description of Artificial Sequence: Synthetic primer
  source 1..22: mol_type = other DNA
tggggccaaatcatgtagactc

SEQ ID NO: 20 - DNA, 22 bp, synthetic construct
  misc_feature 1..22: note = Description of Artificial Sequence: Synthetic primer
  source 1..22: mol_type = other DNA
tcagtgggggtgaattcagtgt

SEQ ID NO: 21 - DNA, 21 bp, synthetic construct
  misc_feature 1..21: note = Description of Artificial Sequence: Synthetic primer
  source 1..21: mol_type = other DNA
cttaacagcgatgctgacccc

SEQ ID NO: 22 - DNA, 22 bp, synthetic construct
  misc_feature 1..22: note = Description of Artificial Sequence: Synthetic primer
  source 1..22: mol_type = other DNA
tggcctccatctcccctctctt